(19) United States
(12) Patent Application Publication
(10) Pub. No.: US 2025/0353898 A1
(43) Pub. Date: Nov. 20, 2025

(54) SERPIN PEPTIDE DERIVATIVES AND METHODS OF USING THE SAME

(71) Applicant: SERPIN PHARMA, LLC, Manassas, VA (US)

(72) Inventors: Cohava Gelber, Nokesville, VA (US); Cyrille Gineste, Manassas, VA (US); Dana Austin, Manassas, VA (US)

(73) Assignee: SERPIN PHARMA, LLC, Manassas, VA (US)

(21) Appl. No.: 18/861,168

(22) PCT Filed: Apr. 27, 2023
(86) PCT No.: PCT/US2023/066321

§ 371 Date: Oct. 28, 2024

Related U.S. Application Data
(60) Provisional application No. 63/363,840, filed on Apr. 29, 2022.

(51) Int. Cl. C07K 14/81

(52) U.S. Cl. CPC C07K 14/8121

(57) ABSTRACT

Of the present technology are SERPIN peptide derivatives, methods of making the same, and uses of the same for treating various conditions associated with LRP1 mediation.

### CROSS-REFERENCE TO RELATED APPLICATION

The present application is a 35 USC § 371 national phase application of Application No. PCT/US2023/066321, filed Apr. 27, 2023, which claims the benefit of, and priority to, U.S. Provisional Patent Application No. 63/363,840 filed Apr. 29, 2022, the disclosures of which are herein incorporated by reference in their entirety.

### SEQUENCE LISTING

This application contains an ST.26 compliant sequence listing, which is being submitted concurrently herewith in .xml format via Patent Center and is hereby incorporated by reference in its entirety. The .xml copy, created on Apr. 26, 2023, is named Serpin 138536-8004WO00 Sequence Listing.xml and is 78,897 bytes in size.

### BACKGROUND

Serine protease inhibitors (SERPINs) are a large family of proteins that are involved in diverse biological functions such as fibrinolysis, blood coagulation and inflammation. When SERPINs bind to their target serine proteases to inactivate the enzymatic activity, a conformational change occurs exposing a unique short peptide motif (5-11 amino acids).^{8,43} The protease-inhibitor complex binds to low-density lipoprotein receptor related protein (LRP1) at the newly exposed short peptide motif, a process which is conserved across the entire spectrum of serine protease inhibitors (SERPINs) such as alpha-1 antitrypsin (AAT) and antithrombin Ill (ATIII).^{22,25,43}

Previously disclosed are a family of SERPIN-derived peptides which were found to bind to LRP1 and exert healing and homeostatic function beyond its anti-inflammatory function. See, e.g., U.S. Pat. Nos. 8,975,224; 9,951,104; 11,020,462; and US Patent Application Publication Nos. 2021/0188912 and 2021/0369822, the contents of which are incorporated herein by reference. The SERPIN-derived peptides such as SP16 and SP163M can be used to treat a number of conditions associated with LRP1 mediation. There is a need to develop novel SERPIN peptide derivatives to further improve the stability, bioavailability, and/or efficacy of the existing SERPIN-derived peptides.

### SUMMARY

In one aspect, of the present technology is a SERPIN peptide derivative comprising, consisting essentially of, or consisting of a pentapeptide having an amino acid sequence of FVFLM (SEQ ID NO: 1), FVFL[Nle] (SEQ ID NO: 2), PFVFLM (SEQ ID NO: 8), or PFVFL[Nle] (SEQ ID NO: 9), and one or more of the following modifications: (i) a polar head added to the N-terminus of the pentapeptide, a polar tail added to the C-terminus of the pentapeptide, or both; (ii) one or more amino acid residues added to the N-terminus of the pentapeptide, C-terminus of the pentapeptide, or both such that the peptide derivative can be cyclized; (iii) one or more amino acid residues in the pentapeptide substituted by one or more amino acid residues having less hydrophobicity; (iv) one or more amino acid residues in the pentapeptide substituted by one or more amino acid residues having greater hydrophobicity; and (v) one or more amino acid residues in the pentapeptide are deleted.

In certain embodiments, the SERPIN peptide derivative of the present technology is a linear peptide. In certain embodiments, the SERPIN peptide derivative of the present technology is a cyclized peptide. In certain embodiments, the SERPIN peptide derivative is cyclized by forming a disulfide bond between two Cys residues. In certain embodiments, the SERPIN peptide derivative is cyclized by a linker between two amino acid residues, for example, two amino acid residues outside the pentapeptide sequence. In certain embodiments, the polar head or the polar tail comprising two or more charged amino acids such as positively charged amino acids selected from the group consisting of Arg, Lys, and His. In certain embodiments, the SERPIN peptide derivative is fused to one or more other peptides include an epitope tag, a half-life extender, or both of an epitope tag and a half-life extender to form a fusion protein or fusion peptide. In certain embodiments, the SERPIN peptide derivative is conjugated to a permeability enhancer.

In another aspect, of the present technology is a composition comprising the SERPIN peptide derivatives, fusions or conjugates of the present technology and one or more pharmaceutically acceptable carriers. In some embodiments, the composition is formulated into a dosage form suitable for oral administration, transdermal administration, or parenteral administration.

In another aspect, of the present technology is a method of treating various conditions or diseases associated with LRP1 binding such as respiratory viral or bacterial infections (e.g., COVID), and inflammatory diseases such as acute respiratory distress, asthma, atopic dermatitis, or eosinophilic esophagitis. Other conditions include those of the central and peripheral nervous system, such as peripheral nerve injury and neurodegenerative disease. The method entails administering an effective amount of one or more SERPIN peptide derivatives, fusions thereof, or a composition comprising one or more SERPIN peptide derivatives or fusions thereof of the present technology to a subject suffers from a condition associated with LRP1 binding.

### BRIEF DESCRIPTION OF THE DRAWINGS

This application contains at least one drawing executed in color. Copies of this application with color drawing(s) will be provided by the Office upon request and payment of the necessary fees.

FIG. 1 shows the crystal structure of α₁-antitrypsin, including a pentapeptide sequence (SEQ ID NO: 8.).

FIG. 2 shows the distance between amino acids based on the crystal structure of α₁-antitrypsin, including a pentapeptide sequence (SEQ ID NO: 8).

FIG. 3 illustrates ring closure strategy using Lys-βAla-Glu 13 C—C bonds as an example.

FIGS. 4A-4B demonstrate the structure-activity relationship of SERPIN-derived peptide derivatives SA1-SA8. FIG. 4A shows the activities in reducing NFκB activation for peptide derivatives SA1-SA8 at various concentrations, 0, 1, 10, 50, and 100 μg/mL. FIG. 4B compares the activities of SA3 and SA7 to those of SP163M and SP22. Compared to the SERPIN peptide SP163M previously disclosed, truncating the peptide while retaining the LRP1 binding site and adding arginine residues conferred improved activity in the NFκB reporter assay.

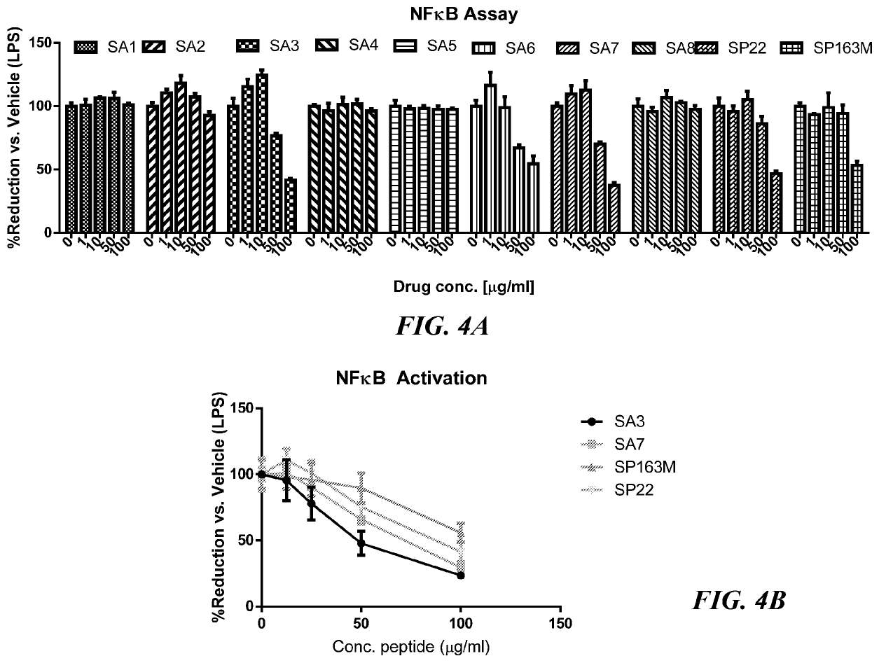

FIGS. 5A-5B demonstrate the anti-inflammatory function of peptide derivatives A1-A15 in comparison to SP163M. The peptide derivatives (50 or 100 μg/ml) were tested in the NFκB reporter assay in response to LPS (5 ng/ml) and screened for TNFα secretion in IMG microglial cells in response to LPS (E. coli 0111:B4) stimulation (100 ng/ml, 24 hours). In the NFκB reporter cells, the NFκB inducible Secreted Embryonic Alkaline Phosphatase (SEAP) was measured in the supernatant. TNFα secretion in the supernatant was measured via ELISA.

FIGS. 6A-6B demonstrate the anti-inflammatory activity of peptide derivatives A2-1 to A2-9 which include modifications to the ring structures and amino acid substitutions. FIG. 6A shows reduction of TNFα in supernatant of IMG microglial cells after activation with LPS (100 ng/ml, 24 hours). FIG. 6B shows the percentage of reduction in NFκB activation following LPS stimulation (5 ng/ml, 24 hours). SP163M, SA7, A5 and A15 peptides were included for comparison.

FIGS. 7A-7B show dose response effects of the peptide derivatives on NFκB and TNFα activation. The peptide derivatives were tested at a concentration of 3.125, 6.25, 12.5, 25, and 50 μg/ml. SP163M, SP16 and SA7 were included at a concentration up to 100 μg/ml. The dose response concentrations are shown in log scale. Peptide derivatives A2-1, A2-2, A2-3, A2-4, and A2-5 exhibited activities superior to SA7, SP16, and SP163M in a dose-dependent manner.

FIG. 8 shows the results of cytotoxicity assay on IMG cells (Microglial). IMG cells were treated with peptide derivatives at concentrations at or exceeding those tested for the activity assays (FIGS. 5-7) for 24 hours. None of the peptides exhibited any cytotoxic effects.

FIG. 9 shows the effects of peptide derivatives A3-1 to A3-16, which include further optimization to the ring structure, on NFκB activation in response to LPS stimulation (5 ng/ml, 24 hours). The peptide derivatives were tested at concentrations between 1.56 μg/ml to 12.5 μg/ml. SP163M and A2-5 were included for comparison. SP163M was tested at concentrations between 12.5 μg/ml and 100 μg/ml. The dose response is shown in log scale.

FIG. 10 shows the activities of peptide derivatives A3-1, A3-2, A3-3, A3-5, A3-6, A3-7, A3-9, A3-14, A3-15, and A3-16 on reducing IL-6 secretion in IMG after LPS stimulation (100 ng/ml, 24 hours). IL-6 secretion was measured by ELISA. Peptide derivative A2-5 and SP163M were included for comparison. SP163M was tested at concentrations between 12.5 and 100 μg/ml. The peptide derivatives including A2-5 were tested at concentrations up to 12.5 μg/ml. The dose response is shown in log scale.

FIGS. 11A-11B show the results of cytotoxicity assay on NFκB reporter cells (FIG. 11A) and IMG microglial cells (FIG. 11B). Peptide derivatives A3-1, A3-2, A3-5, A3-6, A3-7, A3-9, A3-14, A3-15, and A3-16 exhibited no cytotoxic effects when tested at concentrations that were effective in reducing both NFκB and cytokines IL-6 and TNFα (up to 12.5 μg/ml). SP163M was included for comparison and demonstrated no cytotoxic effects at concentrations up to 100 μg/ml. The cells were treated for 24 hours with SP163M or peptide derivatives A3-1, A3-2, A3-5, A3-6, A3-7, A3-9, A3-14, A3-15, A3-16 or A2-5 at concentration indicated.

FIG. 12 shows that peptide derivatives A15 and A2-5 were tested for their ability to block capsaicin induced pain behaviors in mice. Mice were treated with the peptide derivatives by subcutaneous administration 1 hour before capsaicin injection (25 ng). SP163M was administered at a dose of 50 μg/mouse, while the peptide derivatives A15 and A2-5 were administered at a dose of 5 μg/mouse. n=4-9 mice/cohort. One-way ANOVA. *P<0.05, **P<0.01.

FIG. 13 compares the cytokine profile tested in microglial cells treated with either SERPIN peptide derivative SA7 or SERPIN peptide SP163M. SA7 demonstrated significantly increased activity in reducing several cytokines including IL-6 and IL-1β.

FIGS. 14A-14B show that in an LPS induced neuroinflammatory model, the SERPIN peptide derivative SA7 reduced clinical scores (measured daily on a scoring system) (FIG. 14A), and reduced weight loss severity compared to both vehicle (LPS induced) and SP163M (FIG. 14B). SP163M and SA7 were administered by subcutaneous injection at a dose of 100 μg/mouse, 1 hour following LPS administration (1 mg/kg) for four consecutive days. Weights and clinical scores were taken daily, and the brain tissue was collected and processed at the end of the study for analysis. The score key is as follows: 0: bright, alert, responsive; 1-4: slightly scruffy/hunched, less active; 5-10: hunched, lethargic, weight loss; and ≥11 moribund.

FIG. 15 shows that in the neuroinflammatory model of FIG. 14, animals treated with SA7 showed significant differences in the levels of many cytokines measured in the brain lysate, compared to both vehicle and SP163M treated animals, including IL-17A, IL-12, TNFα, and GM-CSF. IL-6 was the most substantially reduced with no detectable levels measured.

FIGS. 16A-16B show that in brain lysate taken from the neuroinflammatory model previously disclosed, the western blot analysis for neurofilament light chain (NfL), a neuronal axon specific marker, and anti-glial fibrillary acidic protein (GFAP), a marker specific to astrocyte activation. The peptide derivative SA7 significantly increased expression of NfL (FIG. 16A), while GFAP expression was decreased significantly (FIG. 16B) compared to vehicle treated and SP163M animals.

FIGS. 17A-17B show that SA7 regulated autophagy during inflammation similar to SERPIN peptide SP163M. Microglial cells were treated with SP163M or SA7 and then exposed to LPS. Western blot analysis of the LC3 II to LC3 1 ratio (initiation of formation of autophagosomes—a biomarkers widely used to detect autophagy) shows that SA7 increased autophagy (FIG. 17A) and LRP1 expression (FIG. 17B) compared to LPS alone, comparable to SP163M. The LRP1 expression was also significantly higher with SA7 without LPS treatment, compared to SP163M.

FIG. 18 shows the protease TMPRSS2 activity and IC50s of SP163M and peptide derivative SA7, with SA7 showing increased inhibition of TMPRSS2 activity in an overexpressing cell system compared to SP163M.

FIGS. 19A-19B demonstrate that LRP1 was decreased in the esophagus of OVA induced mice and SP163M restored LRP1 protein expression. Further, SA7 inhibited the cytokine TSLP production in SPINK7 knockout cells more potently than SP163M and compared to control cells.

FIGS. 20A-20C demonstrate that in a model of OVA induced allergic asthma, peptide derivative A2-5 reduces TSLP, total lung protein and TH-2 mediated cytokines IL-5, IL-4 and IL-13 in the lung homogenate to a greater extent than both OVA induced vehicle treated and SP163M mice.

FIG. 21 illustrates conjugates comprising a peptide derivative of the present technology (e.g., A2-5, SEQ ID NO: 42) and a permeability enhancer.

### DETAILED DESCRIPTION

Of the present technology are SERPIN peptide analogs, and variants and derivatives thereof as well as their uses in prevention or treatment of various conditions by targeting low-density lipoprotein receptor related protein-1 (LRP-1). As used herein, the term “derivative” means a peptide shares amino acid sequence or structure similarity to the pentapeptide FVFLM (SEQ ID NO: 1), FVFL[Nle] (SEQ ID NO: 2), PFVFLM (SEQ ID NO: 8), or PFVFL[Nle] (SEQ ID NO: 9), and contains one or more modifications including insertion, deletion or substitution to improve the stability, bioavailability, and/or biological activities or efficacy compared to the pentapeptide. The terms “derivative,” “variant,” and “analog” may be used interchangeably in this disclosure.

Precise coordination of the immune response is needed to promote inflammatory resolution and mitigate tissue damage and targeting single cytokines or signaling pathways does not resolve all contributing factors in pathology of certain diseases such as those discussed here. A balanced inflammatory response plays a critical role in regeneration and repair and anti-inflammatory drugs have been associated with an opposing action on regeneration and tissue repair.³

SERPIN peptides were previously shown to (1) exert neurotrophic effects, (2) have regenerative and healing properties, (3) show analgesic effects, (4) have anti-viral and anti-microbial properties, and/or (5) exert anti-allergic effects. This combination of activities provides a distinct mechanism in treating conditions associated with peripheral neuropathies such as diabetic peripheral neuropath, degenerative disorders, lung injury, allergic diseases and infectious disease. The SERPIN peptide analogs, and variants and derivatives thereof of the present technology have improved LRP1 binding activity, improved solubility, and/or improved pharmacokinetic properties an oral bioavailability. For example, as demonstrated in the working examples, the SERPIN peptide derivatives of the present technology show improved anti-inflammatory effects and improved efficacy in a model of neuroinflammation.

Accordingly, of the present technology are SERPIN peptide derivatives, pharmaceutical compositions comprising the SERPIN peptide derivatives, and methods of using the same to treat a number of conditions where a dysregulated immune response or impaired endocytic function, or diseases in which LRP1 mediation contributes to pathology, such as in conditions associated with peripheral nerve injury and resulting pain, lung injury, infectious disease and allergic inflammation.

In certain embodiments, the SERPIN peptide derivatives are synthetic peptides. In certain embodiments, the SERPIN peptide derivatives are cyclized. In certain embodiments, the SERPIN peptide derivatives comprise one or more hydrophilic amino acid substitutions. In certain embodiments, the SERPIN peptide derivatives comprise one or more hydrophobic amino acid substitutions. In certain embodiments, the SERPIN peptide derivatives comprise one or more positively charged amino acids at the N-terminus, at the C-terminus, or at both the N-terminus and the C-terminus.

### SERPIN Peptide Derivatives and Modifications

Of the present technology are SERPIN peptide derivatives designed to target LRP1 with a higher affinity to exert more potent anti-inflammatory and cell regenerative effects. These peptide derivatives are modified from the original SERPIN-derived peptides based on structure to activity relationship studies and 3-D modeling of the peptide/LRP1 interaction. These derivatives overcome some of the challenges that are associated with peptide therapeutics such as solubility, plasma stability and oral bioavailability. Compared to the SERPIN peptides previously disclosed such as SP16 (VKFNKPFVFLMIEQNTK) (SEQ ID NO: 4) and SP163M (Ac-VKFNKPFVFL[Nle]IEQNTK-NH₂) (SEQ ID NO: 5), where Nle represents norleucine, the peptide derivatives of the present technology exhibit not only improved LRP1 activity but also improved solubility, pharmacokinetic properties, and bioavailability, in particular, oral and transdermal bioavailability.

It was demonstrated previously that a small peptide fragment of the C-terminal end of alpha-1 antitrypsin (the prototypical SERPIN) was capable of binding to LRP1, exerting potent cell regenerative, tissue protective and immune-modulatory functions. However, the tertiary structure of alpha-1 anti-trypsin (AAT) prevents its binding to LRP1 directly. Rather, AAT can only bind LRP1 when in interaction with its target protease due to a conformational change occurs with AAT that exposes the short 5-11 amino acids binding motif. Surprisingly, the entire highly conserved core sequence VKFNKPFVFLM (SEQ ID NO: 6) is not necessary for the anti-inflammatory effects of the peptide derivatives, as demonstrated by the structure activity relationship studies performed on these derivatives. The derivatives do not contain the FNKP (SEQ ID NO: 7) motif that is highly conserved among SERPINS while retaining the LRP1 binding activity. However, the LRP1 binding motif is highly hydrophobic and unstable in solution, requiring modifications to the SERPIN peptide sequence.

Accordingly, various modifications are made to SP16/SP163M peptides, in particular, in or around the pentapeptide, to produce the SERPIN peptide derivatives with improved properties. As used herein, the “pentapeptide” refers to the FVFLM (SEQ ID NO: 1) sequence in SP16 or FVFL[Nle] (SEQ ID NO: 2) sequence in SPM163, where the Met residue is replaced with a Nle residue. The pentapeptide is responsible for most of the interaction with LRP1. As of the present technology, various modifications are made in the pentapeptide and/or the sequence surrounding the pentapeptide to obtain novel SERPIN peptide derivatives having improved properties. For example, the sequence of the SP163M peptide is further modified by deletion, substitution, and/or cyclization to further improve anti-inflammatory activity, solubility, LRP1 binding activity, and/or oral bioavailability. Comparing to the sequence of SP16 or SP163M, shorter peptide derivatives are developed to achieve better oral bioavailability and brood brain barrier permeability without compromising the anti-inflammatory activity or LRP1 binding activity. In certain embodiments, the peptide derivatives of the present technology have a size of 7, 8, 9, 10, 11, 12, 13, 14, or 15 amino acid residues, preferably, 8, 9, or 10 amino acid residues. For example, the peptide derivatives of the present technology have a size of 8 amino acid residues, 9 amino acid residues, or 10 amino acid residues.

In certain embodiments, a polar head at the N-terminus comprising two or more amino acid residues having a charged side chain, a polar tail at the C-terminus comprising two or more amino acid residues having a charged side chain, or both of a polar head and a polar tail are added to the pentapeptide to improve solubility. In some embodiments, the amino acid residues in the polar head or the polar tail are positively charged and include Arg, His, and Lys. A combination of the same charged amino acid residue or a combination of different amino acid residues can be used for the polar head or the polar tail. For example, the polar head or the polar tail comprises an amino acid sequence of RR, RRR, KK, KKK, HH, HHH, KRR, KR, or RRK. In some embodiments, one or more positively charged amino acid residues in the polar head or the polar tail has a reversed structure. For example, reversed Lys means that the Lys residue is incorporated into the peptide backbone using the carboxylic acid group carried by the α-carbon and the ε-amino group in the side chain rather than both of the amino groups and the carboxylic acid group carried by the α-carbon. Reversed Arg means that the Arg residue is incorporated into the peptide backbone using the guanidinium group carried by the α-carbon rather than the δ-carbon. In some embodiments, two or three Arg residues are added to either or both termini of the pentapeptide. In some embodiments, two or three Arg residues are added to the N-terminus of the pentapeptide.

In certain embodiments, the peptide derivatives of the present technology are cyclized, for example, by forming a disulfide bond between two Cys residues or by a linker between two amino acid residues. As of the present technology, two Cys residues can be added to both termini of the pentapeptide such that a cyclic peptide derivative can be obtained via a disulfide bond. It is within the purview of one of ordinary skill in the art to dispose the Cys residues at a selected location in the peptide derivative to achieve a desired cyclic structure with an optimized ring size. Alternatively, other natural, non-natural, or modified amino acid residues can be added to either or both termini of the pentapeptide such that a linker can be formed between these amino acid residues. The specific amino acid residues can be chosen and disposed at selected locations to achieve a desired cyclic structure with an optimized ring size. Depending on the cyclization strategy such as amide, disulfide, and ring closure metathesis (RCM) or olefin metathesis, amino acid residue substitutions for cyclization can be chosen without significant loss of activity. For example, amino acid residues having a carboxylic acid on its side chain or its C-terminal, including but not limited to Asp, and Glu, or amino acid residues having an amino group on its side chain or its N-terminal, including but not limited to Lys, Dab, and Dap, can be used for amide cyclization, and Cys or any non-natural amino acid carrying a sulfhydryl group on its side chain can be used for —S—S— cyclization. The amino acids can be disposed at any desired locations of the peptide derivatives such that a ring of a desired size can be formed without substantially comprising the activity of the peptide derivative. In some embodiments, a head-to-tail cyclization is formed. In some embodiments, the linker comprises R-Ala. In some embodiments, the linker comprises 2-[(2-amino)-ethoxy]-ethoxy-acetic acid (AEEA). In some embodiments, the ring closing length between the amino acid residues is between 5 and 15 C—C bonds, for example, about 5, about 6, about 7, about 8, about 9, about 10, about 11, about 12, about 13, about 14, or about 15 C—C bonds.

In certain embodiments, the peptide derivatives of the present technology comprise one or more substitutions in the sequence of the pentapeptide to enhance plasma stability and to achieve an increased binding affinity to the peptide's cognate receptor. For example, one or more amino acid residues in the pentapeptide having no interaction or minimal interaction with LRP1 can be replaced by one or more hydrophilic amino acid residues. Additionally, one or more amino acid residues in the pentapeptide interacting with LRP1 can be replaced by one or more amino acid residues having similar but more pronounce physicochemical characteristics. For example, the Phe residue has an aromatic ring on its side chain. The Phe residue can be substituted by Nal (Naphthylalanine) in peptide derivatives 1-5 and 1-6 or Trp which displays a naphthyl or indole ring instead of a phenyl ring. These substitutions greatly improve the aromatic character of the amino acid residue, allowing for more hydrophobic and more aromatic (pi stacking) interaction. In some embodiments, one or more amino acid residues in the pentapeptide can be replaced by one or more natural or non-natural amino acid residues. In some embodiments, one or more amino acid residues in the pentapeptide have a D-configuration. In some embodiments, the side chain of one or more amino acid residues in the pentapeptide is modified. For example, peptide derivative A3-1 comprises a Val to Thr substitution to retain some of the hydrophobicity while introducing some hydrogen bonding, and a Phe to Nal substitution to increase hydrophobic and aromatic interaction. In another example, peptide derivative A3-8 comprises a Nle to D-Ser substitution. Nle does not interact with LRP-1 based on studies of the crystal structure but rather being in the aqueous phase. The side chain of Nle is a hydrophobic linear hydrocarbon chain, which requires energy to solvate it. Replacing the Nle residue by a D-Ser having a hydrophilic side chain facilitates solvation by decreasing the enthalpic penalty which translates into stronger binding energy. Accordingly, in some embodiments, the Nle residue of the pentapeptide is deleted. In some embodiments, one or more of the hydrophobic residues in the pentapeptide are replaced by one or more less hydrophobic residues such as Ala, or by one or more neutral or hydrophilic residues such as Thr and Ser. In some embodiments, one or more of the hydrophobic residues in the pentapeptide are replaced by one or more residues having more hydrophobicity such as Nal. In some embodiments, the Nle residue of the pentapeptide is replaced by an amino acid residue having a D-configuration such as D-Dap, D-Lys, and D-Asp.

The Met or Nle residue in the pentapeptide has a long linear hydrophobic side chain. A substitution of Met or Nle with a hydrophilic amino acid in a D-configuration greatly improves the binding activity of the peptide derivatives to LRP1. Substitutions with amino acid residues having a carboxylic acid in the side chain result in an improvement to some extent, while substitution with an amino acid having a short side chain presenting a hydroxy group (Ser) or an amino group (Dap) achieve the best result.

In certain embodiments, the SERPIN peptide derivatives of the present technology can be further modified to extend the shelf life and/or bioavailability using one or more non-natural peptide bonds or amino acids or by attaching to the peptide functional groups such as polyethylene glycol (PEG).

In certain embodiments, a peptide derivative of the present technology comprises, consists essentially of, or consists of a pentapeptide having an amino acid sequence of FVFLM (SEQ ID NO: 1) or FVFL[Nle] (SEQ ID NO: 2), and a polar head added to the N-terminus of the pentapeptide, a polar tail added to the C-terminus of the pentapeptide, or both. The polar head or the polar tail comprises 2-9 charged amino acid residues such as Arg, Lys, and His. In some embodiments, the polar head or the polar tail comprises 2 or 3 charged amino acid residues. Some examples of the peptide derivatives shown in Table 1 were used to investigate the role of positively charged amino acids in improving solubility and modulating NFκB activation, using SP163M and SP22 as positive controls. The peptide derivatives have various tripeptide sequences added to either or both termini of the LRP1 binding site.

In some embodiments, a peptide derivative comprises, consists essentially of, or consists of a peptide having an amino acid sequence of HHHPFVFLMHHH (SEQ ID NO: 10), HHHPFVFL[Nle]HHH (SEQ ID NO: 11), RRRPFVFL[Nle]RRR (SEQ ID NO: 12), KKKPFVFL[Nle]KKK (SEQ ID NO: 13), EEEVKFNKPFVFL[Nle]EEE (SEQ ID NO: 14), RRRCPFVFL[Nle]CRRR (SEQ ID NO: 15), RRRCPFVFL[Nle]C (SEQ ID NO: 16), CPFVFL[Nle]CRRR (SEQ ID NO: 17), RRRVKFNKPFVFLMRRR (SEQ ID NO: 18), or VKFNKPFVFL[Nle]IEQNTK (SEQ ID NO: 5).

TABLE 1
Peptide Derivatives with
Various Tripeptide Modifications

| | | SEQ |
|---|---|---|
| Peptide | Sequence | ID NO |
| SA1 | Ac-HHHPFVFLMHHH | 10 |
| SA2 | Ac-HHHPFVFL[Nle]HHH | 11 |
| SA3 | Ac-RRRPFVFL[Nle]RRR | 12 |
| SA4 | Ac-KKKPFVFL[Nle]KKK | 13 |
| SA5 | Ac-EEEVKFNKPFVFL[Nle]EEE | 14 |
| SA6 | Ac-RRRCPFVFL[Nle]CRRR | 15 |
| SA7* | Ac-RRRCPFVFL [symbol] Nle [symbol] C(cyclic) | 16 |
| SA8 | Ac-CPFVFL[Nle]CRRR | 17 |
| SP22 | RRRVKFNKPFVFLMRRR | 18 |
| SP163M | Ac-VKFNKPFVFL[Nle]IEQNTK-NH₂ | 5 |

*The underlined sequence denotes cyclization. A disulfide bond is formed between the C residues to cyclize the peptide.

As demonstrated in Example 1, when one or more positively charged amino acids such as arginine (R) residues are added to the LRP1 binding motif, the activity in NFκB reduction is increased. However, addition of one or more other charged amino acids such as positively charged amino acids, e.g., histidine (H) or lysine (K), or negatively charged amino acids, e.g., glutamic acid (E), flanking either side of the LRP1 binding site did not confer activity, indicating the importance and uniqueness of the arginine residues.

In certain embodiments, a peptide derivative of the present technology is a cyclic peptide and comprises, consists essentially of, or consists of a pentapeptide having an amino acid sequence of FVFLM (SEQ ID NO: 1) or FVFL[Nle] (SEQ ID NO: 2), and a polar head added to the N-terminus of the pentapeptide, a polar tail added to the C-terminus of the pentapeptide, or both. The cyclization can be between any two residues, for example, the cyclization can be a head-to-tail cyclization. Additional residues such as Cys can be inserted to facilitate the formation of S—S bond to connect two residues. In some embodiments, one or more hydrophobic residues of F, M, or Ne in the pentapeptide are substituted with a neutral or hydrophilic residue. Accordingly, peptide derivatives having various combinations of positively charged amino acid residues addition and/or amino acid substitutions, some examples shown in Table 2, were designed to improve solubility, stability, and/or oral bioavailability without compromising their NFκB modulating activities.

In certain embodiments, a peptide derivative of the present technology comprises a pentapeptide having an amino acid sequence of FVFLM (SEQ ID NO: 1) or FVFL[Nle] (SEQ ID NO: 2), and an additional proline (“P”) amino acid residue. In some aspects, the P amino acid residue is at the C-terminal end of the pentapeptide, such that the peptide derivative comprises an amino acid sequence of PFVFLM (SEQ ID NO: 8) or PFVFL[Nle] (SEQ ID NO: 9). In some aspects, the peptide derivative comprising an amino acid sequence of PFVFLM (SEQ ID NO: 8) or PFVFL[Nle] (SEQ ID NO: 9) may be a cyclized peptide. In some aspects, the peptide derivative comprising an amino acid sequence of PFVFLM (SEQ ID NO: 8) or PFVFL[Nle] (SEQ ID NO: 9) may by cyclized by further comprising Cys residues to facilitate the formation of S—S bonds, for example a peptide derivative having an amino acid sequence of CPFVFLMC (SEQ ID NO: 19) or CPFVFL[Nle]C (SEQ ID NO: 20). In some aspects, the peptide derivative having a sequence of CPFVFLMC (SEQ ID NO: 19) or CPFVFL[Nle]C (SEQ ID NO: 20) may further comprise a polar head, for example three R amino acid residues. In some aspects, the peptide derivative may comprise a sequence of RRRCPFVFLMC (SEQ ID NO: 21) or RRRCPFVFL[Nle]C (SEQ ID NO: 22). In some aspects, the peptide derivative having a sequence of RRRCPFVFLMC (SEQ ID NO: 21) or RRRCPFVFL[Nle]C (SEQ ID NO: 22) may be further acetylated. In some aspects, the peptide derivative may be peptide SA7.

In certain embodiments, a peptide derivative of the present technology comprises, consists essentially of, or consists of an amino acid sequence of RCFVFL[Nle]C (SEQ ID NO: 23), RRCFVFL[Nle]C (SEQ ID NO: 24), RRCFVFL[Nle]C (SEQ ID NO: 25), RRRCFVFL[Nle]C (SEQ ID NO: 26), RRRCFVFT[Nle]C (SEQ ID NO: 27), RRRCFTFL[Nle]C (SEQ ID NO: 28), or RRRCTVFL[Nle]C (SEQ ID NO: 29).

TABLE 2
Peptide Derivatives to Improve Solubility

| | | | SEQ |
|---|---|---|---|
| Peptide | Sequence | Structure | ID NO |
| A1* | Guar-RCFVFL[Nle]C (cyclic) | 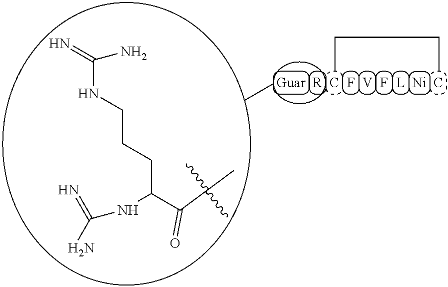 | 23 |
| A2* | RRCFVFL[Nle]C (cyclic) | 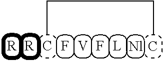 | 24 |
| A3* | Ac-RRCFVFL[Nle]C (cyclic) | 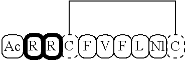 | 25 |
| A4* | Ac-RRRCFVFL[Nle]C (cyclic) | 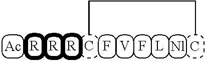 | 26 |
| A7* | Ac-RRRCFVFT[Nle]C (cyclic) | 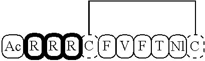 | 27 |
| A8* | Ac-RRRCFTFL[Nle]C (cyclic) | 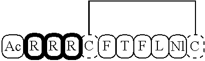 | 28 |
| A9* | Ac-RRRCTVFLINle]C (cyclic) | 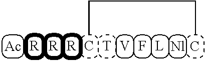 | 29 |

*The underlined sequence denotes cyclization. A disulfide bond is formed between the C residues to cyclize the peptide.

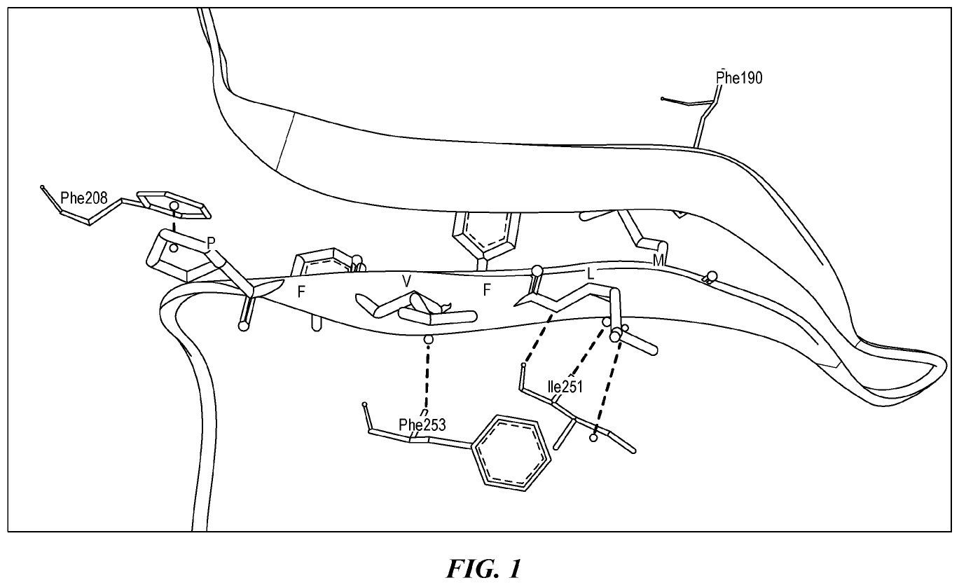

As shown in FIG. 1, F, F, and M residues of the pentapeptide FVFLM (SEQ ID NO: 1) point to one direction, while the V and L residues point to the opposite direction. The P residue at the N-terminus of the pentapeptide creates a turn in the structure but has no interaction with LRP1. It is unlikely that all five residues of the pentapeptide directly interact with LRP1. Additionally, peptide derivatives A7-A9 are designed to replace one of the hydrophobic amino acid residues (F, V, and L) with a residue having a higher hydrophilicity such as T to investigate whether the solubility and/or activity can be improved.

Table 3 shows some examples of the peptide derivatives having one of the F residues replaced by 1-naphylalanine, which is a non-natural amino acid with an additional aromatic ring. The naphylalanine (Nal) substitution increases hydrophobicity and may have stronger hydrophobic interaction with LRP1.

In certain embodiments, a peptide derivative of the present technology comprises, consists essentially of, or consists of an amino acid sequence of

(SEQ ID NO: 30)
RRRCFV[Nal]L[Nle]C
or
(SEQ ID NO: 31)
RRRC[Nal]VFL[Nle]C.

TABLE 3
Peptide Derivatives with Substitutions to Improve Activity

| | | | SEQ ID |
|---|---|---|---|
| Peptide | Sequence | Structure | NO: |
| A5* | Ac- RRRCFV[Nal]L[Nle]C (cyclic) | 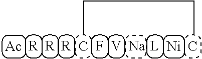 | 30 |
| A6* | Ac- RRRC[Nal]VFL[Nle]C (cyclic) | 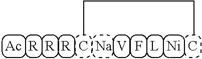 | 31 |

*The underlined sequence denotes cyclization. A disulfide bond is formed between the C residues to cyclize the peptide.

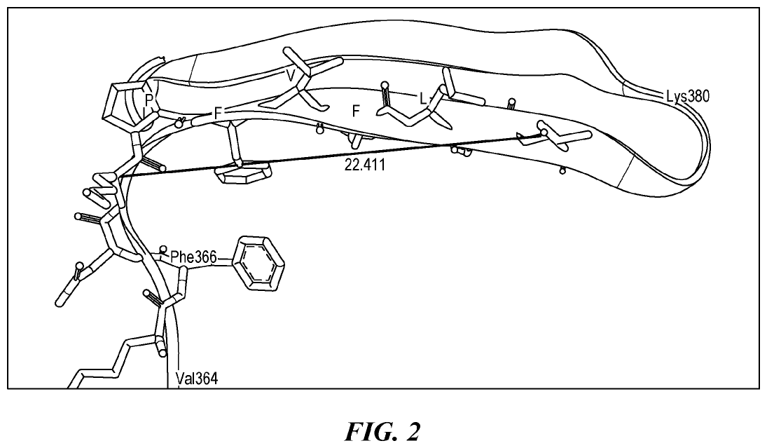

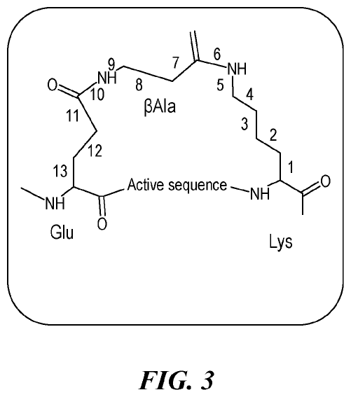

Additional peptide derivatives were designed to determine the optimal ring size. For example, the Cys-Cys bridge can be replaced by a linker of a greater length to improve cyclization. Based on the crystal structure of the LRP1 binding site, the distance between the α-carbon of the amino acid on the N-terminus and the α-carbon of the amino acid on the C-terminus of the pentapeptide FVFLM (SEQ ID NO: 1) was measured. Assuming 1.5 Å for C—C bond's length, the distance between the amino acids is converted to a number of C—C bonds which can be used to design a cyclization strategy. For example, the distance between the α-carbons of K368 and 1375 is 22.4 Å (approximately 15 C—C bonds) and between the α-carbons of P369 and 1375 is 20.2 Å (approximately 13.5 C—C bonds). See FIG. 2. To cyclize the derivative and mimic the distance between P369 and 1375, a linker of between 13 to 14 C—C bonds using a β-Ala can be used to connect the side chain of a Lys residue to the side chain of a Glu residue to cyclize the peptide derivative with a 13 C—C bond ring closing length. See FIG. 3.

In certain embodiments, a peptide derivative of the present technology comprises, consists essentially of, or consists of an amino acid sequence of

(SEQ ID NO: 32)
RRRDFVFL[Nle][Dap],
(SEQ ID NO: 33)
RRRDFVFL[Nle][Dap],
(SEQ ID NO: 34)
RRRDFVFL[Nle][Dap],
(SEQ ID NO: 35)
RRREFVFL[Nle]K,
(SEQ ID NO: 36)
RRREFVFL[Nle]K,
(SEQ ID NO: 37)
RRRFVFL[Nle]D.

Table 4 illustrates some examples of peptide derivatives having various ring closure designs where a ring closing linker is used to replace the Cys-Cys bridge.

TABLE 4
Peptide Derivatives with Various Ring Closure

| | | | | SED |
|---|---|---|---|---|
| | | | | ID |
| Peptide | Sequence | Structure | Ring Closure | No |
| A10* | Ac- RRRDFVFL[Nle][Dap] (cyclic) | 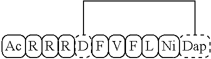 | Linker between Asp and 2,3- diaminopropionic acid (Dap) | 32 |
| | | | having a ring | |
| | | | closing length of | |
| | | | 5 C-C bonds. | |
| A11* | Ac- RRRDFVFL[Nle][Dap] (cyclic) | 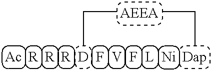 | Asp-2-[(2- amino)-ethoxy]- ethoxy-acetic acid (AEEA)- Dap linker | 33 |
| | | | having a ring | |
| | | | closing length of | |
| | | | 13 C-C bonds. | |
| A12* | Ac- RRRDFVFL[Nle]Dap] (cyclic) | 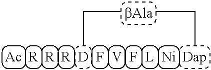 | Asp-ßAla-Dap linker having a ring closing length of 8 C-C bonds. | 34 |
| A13* | Ac-RRREFVFL[Nle]K (cyclic) | 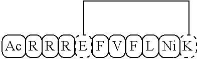 | Linker between Glu and Lys having a ring closing length of | 35 |
| | | | 10 C-C bonds. | |
| A14* | Ac-RRREFVFL[Nle]K (cyclic) | 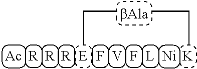 | Glu-ßAla-Lys linker having a ring closing length of 13 C-C bonds. | 36 |
| A15* | HN-RRRFVFL[Nle]D (cyclic) | 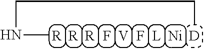 | Head-to-tail linker between the amino group | 37 |
| | | | of the N-terminal | |
| | | | amino acid Arg | |
| | | | and the | |
| | | | carboxylic acid | |
| | | | on the side | |
| | | | chain of the C- | |
| | | | terminal amino | |
| | | | acid, Asp, | |
| | | | having a ring | |
| | | | closing length of | |
| | | | 12 C-C bonds. | |

*The underlined sequence denotes cyclization.

Example 2 demonstrates that peptide derivatives A5, A8, A10, and A15 exhibited improved activities in reducing TNFα activation. Table 5 lists some examples of additional SERPIN peptide derivatives having similar modifications, as well as peptide derivatives having substitutions in the pentapeptide. For example, NMe in the pentapeptide is substituted with Ala or deleted to determine whether NMe is involved in the interaction with LRP1. Additionally, each residue of the pentapeptide is substituted with D-Ser (dS) to improve solubility and resistance to protease.

In certain embodiments, a peptide derivative of the present technology comprises, consists essentially of, or consists of an amino acid sequence of

(SEQ ID NO: 38)
RRR[Dap]FT[Nal]L[Nle]D,
(SEQ ID NO: 39)
RRRFT[Nal]L[Nle]D,
(SEQ ID NO: 40)
RRRFVFLAD,
(SEQ ID NO: 41)
RRRFVFLD,
(SEQ ID NO: 42)
RRRFVFL[dS]D,
(SEQ ID NO: 43)
RRRFVF[dS][Nle]D,
(SEQ ID NO: 44)
RRRFV[dS]L[Nle]D,
(SEQ ID NO: 45)
RRRF[dS]FL[Nle]D,
or
(SEQ ID NO: 46)
RRR[dS]VFL[Nle]D.

TABLE 5
Peptide Derivatives with Various Ring Closure and AA Substitutions

| | | | SEQ ID |
|---|---|---|---|
| Peptide | Sequence | Structure | NO |
| A2-1* | Ac- RRR[Dap]FT[Nal]L[Nle]D (cyclic) | 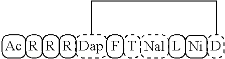 | 38 |
| A2-2* | HN-RRRFT[Nal]L[Nle]D (cyclic) | 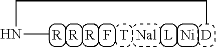 | 39 |
| A2-3* | HN-RRRFVFLAD (cyclic) | 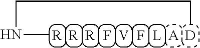 | 40 |
| A2-4* | HN-RRRFVFLD (cyclic) | 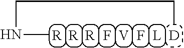 | 41 |
| A2-5* | HN-RRRFVFL[dS]D (cyclic) | 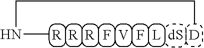 | 42 |
| A2-6* | HN-RRRFVF[dS][Nle]D (cyclic) | 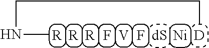 | 43 |
| A2-7* | HN-RRRFV[dS]L[Nle]D (cyclic) | 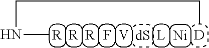 | 44 |
| A2-8* | HN-RRRF[dS]FL[Nle]D (cyclic) | 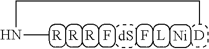 | 45 |
| A2-9* | HN-RRR[dS]VFL[Nle]D (cyclic) | 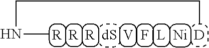 | 46 |

*The underlined sequence denotes cyclization.

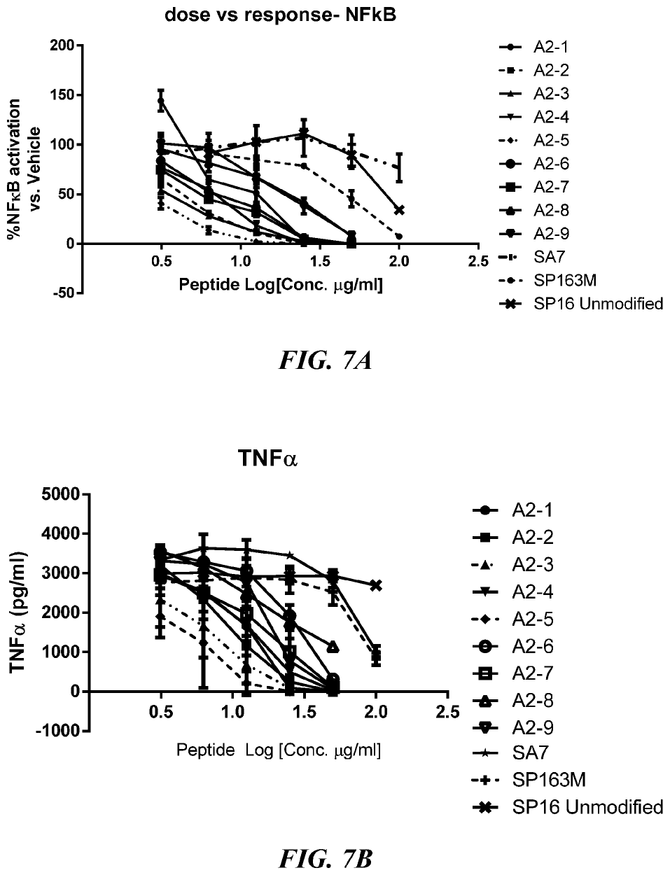

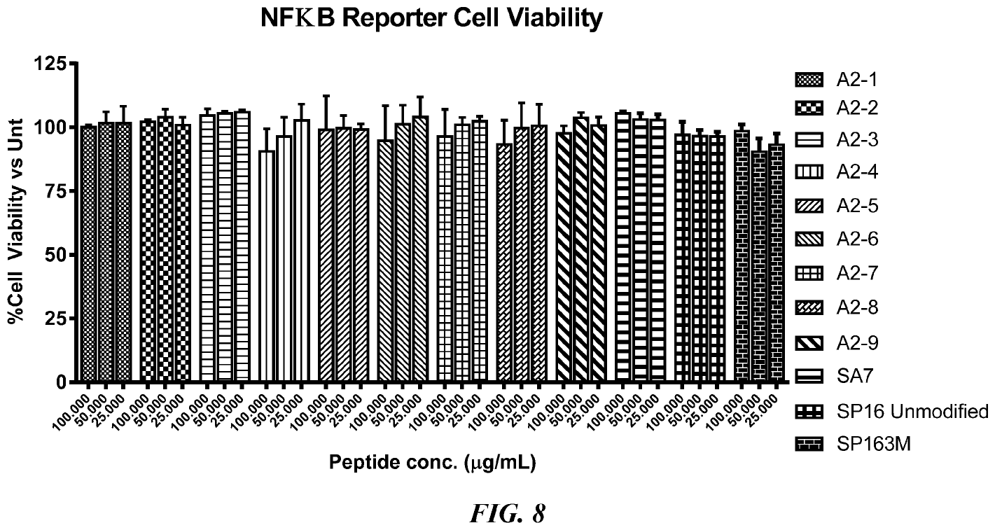

As shown in FIG. 7, peptide derivatives A2-1, A2-2, A2-3, A2-4, and A2-5 demonstrated superior activities to SP163M and SA7 in reducing TNFα and NFκB activation. None of the tested peptide derivatives exhibited any significant toxicity on the cell lines tested (FIG. 8).

In certain embodiments, a peptide derivative of the present technology comprises, consists essentially of, or consists of an amino acid sequence of

(SEQ ID NO: 47)
RRRFT[Nal]LD,
(SEQ ID NO: 48)
[rK]RRFT[Nal]LD,
(SEQ ID NO: 49)
[rR]RRFT[Nal]LD,
(SEQ ID NO: 50)
[rR][rR]FT[Nal]LD,
(SEQ ID NO: 51)
RRR[Dap]FT[Nal]LD,
(SEQ ID NO: 52)
RR[Dap]FT[Nal]LD,
(SEQ ID NO: 53)
RRRKFT[Na]LD,
(SEQ ID NO: 54)
RR[Dap]FT[Nal]L[dS]D,
(SEQ ID NO: 56)
RRRFVFL[dDap]D,
(SEQ ID NO: 57)
RRRFVFL[dK]D,
(SEQ ID NO: 58)
RRRFVFL[dD]D,
(SEQ ID NO: 59)
RRRFTF[Nal]A[Nle]D,
(SEQ ID NO: 60)
RRRAT[Nal]L[Nle]D,
(SEQ ID NO: 61)
RRRFT[Nal]L[dS]D,
(SEQ ID NO: 62)
RRRFTWL[dS]D.

Table 6 provides examples of SERPIN peptide derivatives that are further optimized by shortening the peptide length via deletion, optimizing the cyclization, and/or residue substitutions to further improve solubility, activities, and/or oral availability. In certain embodiments, one or more amino acid residues in the pentapeptide of the peptide derivative are deleted or substituted with one or more natural or non-natural amino acid residues. For example, Ne residue can be substituted by a less hydrophobic amino acid such as Ala or Ser (D-Ser) to improve the agonist activity binding to LRP1. In certain embodiments, amino acids of D-configuration may be used to change the orientation of the amino acid in the 3-D structure of the peptide and/or to confer protease stability. In certain embodiments, two charged amino acid residues such as Arg and/or Lys are added to the N-terminus of the pentapeptide. In certain embodiments, three charged amino acid residues such as Arg and/or Lys are added to the N-terminus of the pentapeptide. The charged amino acid(s) can have a “reversed” chemical structure to optimize the ring size, as illustrated in the examples below:

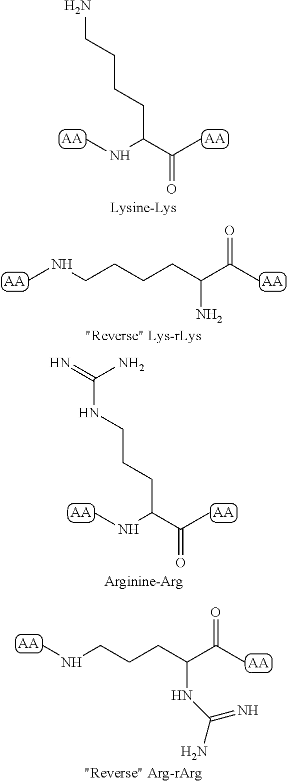

TABLE 6
Peptide Derivatives with Additional Modifications

| | | | | SEQ |
|---|---|---|---|---|
| Peptide | Sequence | Structure | Modification | ID NO |
| A3-1* | HN- RRRFT[Nal]]LD (cyclic) | 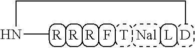 | Optimized pentapeptide sequence with Nle | 47 |
| | | | deleted in a head to tail | |
| | | | cyclization. | |
| A3-2* | HN- [rK]RRFT[Nal]]LD (cyclic) | 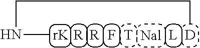 | Reverse Lys cyclization from E- amino group on lysine | 48 |
| | | | side chain to carboxylic | |
| | | | acid of Asp to obtain | |
| | | | same ring size as the | |
| | | | derivative having Nle. | |
| A3-3* | HN- [rR]RRFT[Nal]]LD (cyclic) | 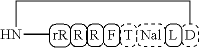 | The a-amino of the reverse lysine of A3-2 is guanylated to obtain | 49 |
| | | | rArg to improve | |
| | | | solubility. | |
| A3-4* | HN- [rR][[rR]FT[Na]]LD (cyclic) | 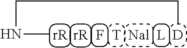 | Two rArg are used to while deleting one Arg and Nle to conserve | 50 |
| | | | the ring size and | |
| | | | activity. | |
| A3-5* | Ac- RRR[Dap]FT[Nal]LD (cyclic) | 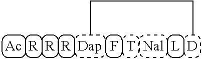 | Optimized pentapeptide sequence with Nle deleted in a Dap-Asp | 51 |
| | | | cyclization. | |
| A3-6* | Ac- RR[Dap]FT[Nal]LD (cyclic) | 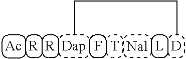 | One Arg residue is deleted to shorten the peptide. | 52 |
| A3-7* | Ac- RRRKFT[Nal]]LD (cyclic) | 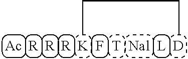 | Lys-Asp cyclization is used to compensate for the loss of a ring size of approximately 3 | 53 |
| | | | C-C bonds due to | |
| | | | deletion of Nle. | |
| A3-8* | Ac- RR[Dap]FT[Nal]L[dS]D (cyclic) | 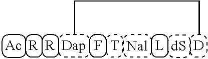 | A D-Ser residue is added to improve solubility. | 54 |
| A3-9* | Ac- RR[Dap]FT[Nal]L[dS]D (cyclic) | 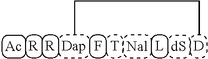 | Cyclization is done using a secondary amine instead of an amide bond to add an | 54 |
| | | | extra basic center to | |
| | | cyclization with amine | increase solubility. | |
| A3-10* | HN- RRRFVFL[dDap]D (cyclic) | 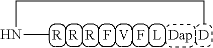 | Substitution with D- Dap, the side chain of which has the same | 56 |
| | | | shape and length as | |
| | | | Ser but displays an | |
| | | | amino group instead of | |
| | | | an hydroxy group to | |
| | | | increase hydrophilicity. | |
| A3-11* | HN- RRRFVFL[dK]D (cyclic) | 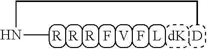 | The side chain of D- Lys carries an amino group but is further | 57 |
| | | | away from the peptide | |
| | | | backbone. | |
| A3-12* | HN- RRRFVFL[dD]D (cyclic) | 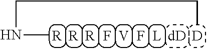 | D-Asp side chain has the same shape and length as Ser but | 58 |
| | | | displays a carboxylic | |
| | | | acid group instead of a | |
| | | | hydroxy group to | |
| | | | increase hydrophilicity. | |
| A3-13* | HN- RRRFTF[Nal]A[Nle]D (cyclic) | 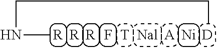 | Leu is substituted by Ala. | 59 |
| A3-14* | HN- RRRAT[Nal]L[NIe]D (cyclic) | 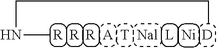 | Phe is substituted by Ala. | 60 |
| A3-15* | HN- RRRFT[Nal]L[dS]D (cyclic) | 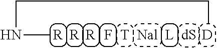 | Phe is substituted by a non-natural amino acid, Nal, to gain | 61 |
| | | | hydrophobic | |
| | | | interaction at this | |
| | | | position. | |
| A3-16* | HN- RRRFTWL[dS]D (cyclic) | 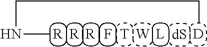 | Phe is substituted by a natural amino acid, Trp, to gain | 62 |
| | | | hydrophobic | |
| | | | interaction at this | |
| | | | position. | |

*The underlined sequence denotes cyclization.

The peptide derivatives of the present technology can be further modified in the amide bonds to improve protease stability and absorption, and these modifications include but are not limited to peptide bond isostere, N-methylation, and/or D-configuration amino acid substitution.

In certain embodiments, disclosed is a peptide derivative comprising, consisting essentially of, or consisting of the amino acid sequence X1-X2-X3-X4-X5-X6-X7-X8-X9-X10-X11 (SEQ ID NO: 3), wherein:
X1 is a hydrophilic amino acid residue or none;
X2 is a hydrophilic amino acid residue or none;
X3 is a hydrophilic amino acid residue or none;
X4 is a Cys amino acid residue or none;
X5 is a Pro amino acid residue or none;
X6 is a first hydrophobic amino acid residue;
X7 is a short-branched amino acid residue;
X8 is a second hydrophobic amino acid residue;
X9 is a saturated hydrophobic amino acid residue;
X10 is a hydrophilic amino acid of D configuration; and
X11 is any amino acid residue which allows cyclization of the peptide derivative.

In some embodiments, SEQ ID NO: 3 further comprises one or more of the following: X4 is Cys, X5 is Pro, X7 is Thr, and X10 is a D-configured Lys residue.

In some embodiments, the first and second hydrophobic amino acid residues of X6 and X8 are aromatic amino acid residues. In some embodiments, the short-branched amino acid residue of X7 is Val or Thr. In some embodiments, the saturated hydrophobic amino acid of X9 is Leu. In some embodiments, the hydrophilic amino acid of D configuration of X10 is a D-configured Asp, Glu, Lys, Dap, or Cys residue.

In some embodiments, X1 is a basic residue displaying a positive charge or none; X2 is a basic residue displaying a positive charge or none; and X3 is a basic residue displaying a positive charge or none. In some embodiments, X1 is a Arg, Lys or His residue or none; X2 is a Arg, Lys or His residue or none; and X3 is a Arg, Lys or His residue or none.

In some embodiments, X6 is Phe or Nal. In some embodiments, X8 is Phe or Nal. In some embodiments, X6 is Ala, Phe, or Nal, and X4 is Nal. In some embodiments, X7 is Asp, Glu, Lys, Dap, or Cys.

In some embodiments, the peptide derivative is linear or cyclized. In some embodiments, the peptide derivative has a size of less than 15 amino acid residues. In some embodiments, the peptide derivative has a size of 7, 8, 9, 10, 11, or 12 amino acid residues.

In certain embodiments, the peptide derivatives of the present technology acetylated at the N-terminus, amidated at the C-terminus, or both.

In certain embodiments, the peptide derivative is cyclized with 10 C—C bonds or equivalent to close the ring between the α-carbon of X4 and X11 of SEQ ID NO: 3. Cyclization can also occur between X11 and an extra amino acid residue added between X4 and X5 of SEQ ID NO: 3. Cyclization can also occur between X11 and an extra amino acid residue added between X5 and X6 of SEQ ID NO: 3. In this situation the optimal ring is closed using 5 C—C bonds (or equivalent). In certain embodiments, the cyclization is a head-to-tail cyclization.

In certain embodiments, the SERPIN peptide derivatives of the present technology has a size of between 5 and 30 amino acids, for example, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, the SERPIN peptide derivative of the present technology has a size of 20 amino acids or less. A longer peptide may have a decreased solubility, whereas a shorter peptide may have decreased stability. In certain embodiments, the peptide derivative has a size of 9 amino acid residues; however, a peptide derivative having a size of 8 amino acid residues in conjunction with an optimized cyclization strategy suffers only minimal activity loss.

In certain embodiments, the SERPIN peptide derivatives of the present technology are fused to one or more other peptides or compounds to form a fusion peptide or fusion protein. For example, one or more other peptides include an epitope tag such as ALFA-tag, V5-tag, Myc-tag, HA-tag, Spot-tag, T7-tag, NE-tag, a half-life extender such as poly(ethylene glycol) (PEG), Lipidation, FC fusion, or Albumin fusion, or both of an epitope tag and a half-life extender.

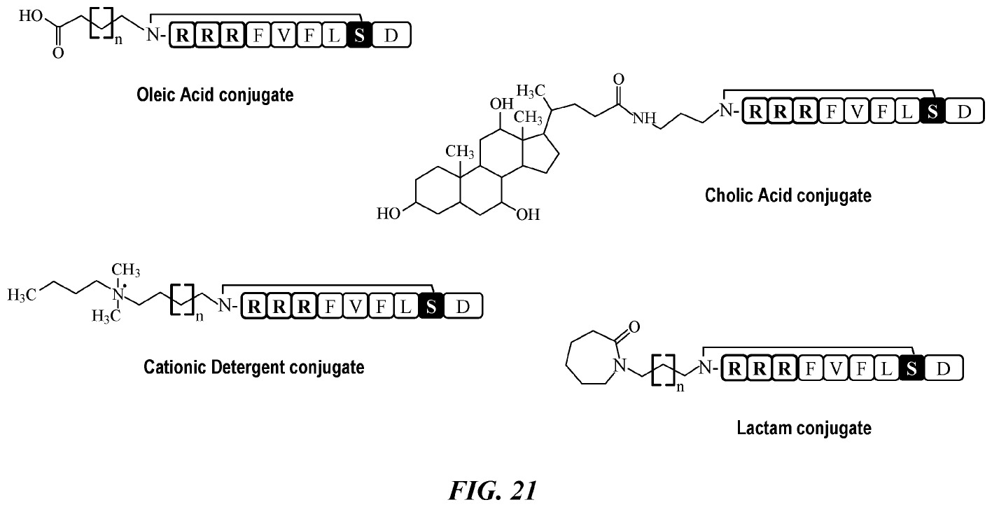

In certain embodiments, the peptide derivatives of the present technology can be conjugated to a permeability enhancer such as oleic acid, cholic acid, a cationic detergent, or lactam to further improve the transdermal, trans-epithelial, nasal, gastric or topical bioavailability, as illustrated in FIG. 21.

### Pharmaceutical Compositions Comprising the SERPIN Peptide Derivatives

In another aspect, this disclosure relates to a pharmaceutical composition comprising, consisting essentially of, or consisting of an effective amount of one or more SERPIN peptide derivatives or fusion peptides of the present technology. The pharmaceutical compositions of the present technology can be formulated into any suitable dosage form for transdermal, sublingual, nasal, oral, inhalation, rectal or ocular administration. In some embodiments, the pharmaceutical composition further comprises one or more additional therapeutic agents, which are not the SERPIN peptide derivatives of the present technology. In some embodiments, the pharmaceutical composition further comprises one or more permeability enhancers in an amount of 0.1 to 5% (w/w) to promote penetration of the peptide derivative or the fusion peptide into skin, mucosal membrane, nasal mucosa, or an epithelial layer. Non-limiting examples of permeability enhancers include a fatty acid such as oleic acid, a bile acid such as cholic acid, a cationic detergent such as cetyltrimethylammonium, and a lactam such as laurocapram. In some embodiments, the pharmaceutical composition further comprises a pharmaceutically acceptable carrier, excipient, additive, preservative, or a combination thereof. Examples of acceptable carriers include physiologically acceptable solutions, such as sterile saline and sterile buffered saline.

The term “an effective amount” as used herein refers to an amount of a composition that produces a desired effect. An effective amount of a composition may be used to produce a prophylactic or therapeutic effect in a subject, such as preventing or treating a target condition, alleviating symptoms associated with the condition, or producing a desired physiological effect. In such a case, the effective amount of a composition is a “therapeutically effective amount,” “therapeutically effective concentration” or “therapeutically effective dose.” The precise effective amount or therapeutically effective amount is an amount of the composition that will yield the most effective results in terms of efficacy of treatment in a given subject or population of cells. This amount will vary depending upon a variety of factors, including but not limited to the characteristics of the composition (including activity, pharmacokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further an effective or therapeutically effective amount may vary depending on whether the composition is administered alone or in combination with another composition, drug, therapy or other therapeutic method or modality. One skilled in the clinical and pharmacological arts will be able to determine an effective amount or therapeutically effective amount through routine experimentation, namely by monitoring a cell's or subject's response to administration of a composition and adjusting the dosage accordingly. For additional guidance, see Remington: The Science and Practice of Pharmacy, 21^{st} Edition, Univ. of Sciences in Philadelphia (USIP), Lippincott Williams & Wilkins, Philadelphia, PA, 2005.

In certain embodiments, the peptide derivatives or the pharmaceutical compositions of the present technology may be formulated for oral administration, parenteral administration such as intravenous administration, intramuscular administration, subcutaneous administration (bolus injection or through a device such as an infusion pump), intradermal administration, transdermal administration, topical administration, and intranasal administration. In certain embodiments, a subcutaneous infusion pump can be used for delivery of the peptides or the pharmaceutical compositions of the present technology. The peptides or the pharmaceutical compositions may be administered more than once. More specifically, after the initial administration, one or more additional doses may be given as a booster.

The SERPIN peptide derivatives, fusion peptides, or the pharmaceutical compositions of the present technology have various functions. In certain embodiments, of the present technology is a method of treating a subject in need thereof an effective amount of one or more SERPIN peptide derivatives, fusion peptides, or the pharmaceutical compositions disclose herein. In some embodiments, the subject suffers from a disease or condition in which LRP1 mediation contributes to pathology, such as in conditions associated with peripheral nerve injury and resulting pain, lung injury, infectious disease and allergic inflammation such as eosinophilic esophagitis. In some embodiments, the subject suffers from a disease associated with dysregulated immune response selected from the group consisting of peripheral neuropathies, neuropathic pain, COVID-19 infection, acute respiratory distress syndrome (ARDS), sepsis, SARS-CoV-2, Influenza, alphavirus infection, and cytokine storm.

As used herein, “treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. Treatment may also mean a prophylactic or preventative treatment of a condition.

As used herein, the term “subject” is an animal. In some embodiments, the subject is a mammal. In some embodiments, the subject is human. In some embodiments, the subject has not received any prior treatment with serine protease inhibitors, such as alpha-1-antitrypsin treatment before the treatment with the peptides of the present technology.

In some embodiments, the peptides can be used to reduce the serum TNF-α levels in human individuals who have pathologically increased TNF-α levels. The peptide causes a 75% decrease in serum TNF-α levels when administered in an effective amount to a human subject. In certain embodiments, the peptide results in a 50% or 75% decrease in serum TNF-α levels when administered in an effective amount to a human subject compared to the levels before administration of the peptide.

Conditions Associated with LRP-1

LRP-1 functions as an endocytic and cell signal transduction receptor and has several ligands that induce specific cell signaling cascades that can contribute to cell survival and anti-inflammatory mechanisms.^{5,18,22,25} LRP1 is ubiquitously expressed on many different organs, abundantly in brain, lung, heart and immune cells. Because of these unique capabilities and wide expression on both tissues and immune cells, it plays a critical role in regulating inflammation, cellular metabolism, and maintaining homeostasis. For instance, LRP1 regulates inflammatory signaling pathways such as NFκB and JNK pathways that induce the conversion of pro-inflammatory (M1) macrophages to the anti-inflammatory (M2) macrophage phenotype, regulates the cytokine output, and contributes to effective migration and phagocytosis.^{22,26,51} In neutrophils, LRP1-dependent mechanisms lead to enhanced cell adhesion, chemotaxis, and antibacterial effects of these cells, thereby resisting immunosuppression²⁵. During acute infection or injury, LRP1 also promotes inflammatory resolution through scavenging PAMPS and DAMPS from dying or injured tissue, to prevent the tissue injury cycle²⁵. LRP1 was also shown to mediate autophagy during infection, an important metabolic process recently shown to play an important protective role in a variety of diseases.^{4,10} Therefore, because of its multifunctional ability to regulate inflammation, targeting LRP1 has substantial potential to mitigate several aspects of the immune response that contributes to the pathology of several diseases including neurological disorders, infectious diseases, and allergic inflammatory disease.

### Neurological Disease

In terms of nerve injury and associated pain, injury to the peripheral nervous system induces an increase in the expression of LRP1. Previously, it was demonstrated that LRP1 agonist are capable of promoting axonal growth in the CNS and are capable of inducing regeneration after spinal cord injury.⁵³ LRP1 is an endocytic receptor to a diverse number of ligands including tissue-type plasminogen activator (tPA), matrix metalloproteinase-9 (MMP-9), and activated α2-macroglobulin.¹⁴ These ligands are capable of inducing anti-inflammatory activity,³⁹ activating the Schwann cell repair program²¹ and transactivation of cell signaling pathways in neurons associated with axonal regeneration.³⁸ LRP1 requires ligand-binding to activate cell-signaling, however, different ligands elicit distinct and sometimes opposing cell-signaling responses reflecting the ability of different ligands to assemble unique co-receptor complexes. Furthermore, many LRP1 ligands are multi-domain proteins with numerous effects on cell physiology that do not involve LRP1-binding. For example, tissue-type plasminogen activator (tPA) binds to LRP1 to promotes Schwann Cell (SC) survival and migration.²³ Yet, by LRP1-independent activities, tPA elicits pain.¹ El-tPA promotes survival of human iPSC-derived neural progenitor cells (iNPCs) and transplanted El-tPA activated iNPCs into rodents with severe spinal cord injury demonstrate improved motor functional recovery.⁴⁰ Imbalances in the microenvironment following nerve injury may have severe consequences, including the development of chronic neuropathic pain states.¹² In peripheral nervous system (PNS) injury, both inflammatory cytokines such as TNFα, IL-6 and IL-1β and anti-inflammatory cytokines such as IL-10 have been shown to play a central role in axon regeneration and repair.⁶

In terms of neurodegenerative disease such as Alzheimer's Disease (AD), LRP1's role has been extensively studied. AD is characterized by a progressive loss of cognitive abilities and formation of senile plaques, which are composed largely of amyloid β (Aβ), and tau protein aggregates called neurofibrillary tangles (NFTs) in the hippocampus and cortex of afflicted humans. The spread of protein aggregates during disease progression is a common theme underlying neurodegenerative disease pathology. As an endocytic receptor, LRP1 was shown to regulate brain and systemic clearance, degradation and production of amyloid β-peptide.⁵⁴ As LRP1-mediated clearance of Aβ across the blood brain barrier (BBB) is the key event in the regulation of Aβ transcytosis from brain to periphery, targeting LRP1 with one or more peptide derivatives of the present technology may serve as a novel treatment. Also, tau protein aggregates forming NTFs plays a central role in the pathogenesis of Alzheimer's disease. LRP1 functions to regulate tau protein endocytosis, accumulation and spread associated with worsened pathology.³² Therefore, the SERPIN peptide derivatives of the present technology may serve as an intervention that remediates both plaque and tangle pathologies through mediation of LRP1 associated protein aggregation of tau and amyloid a β. The SERPIN peptide derivatives of the present technology can also act on neuroinflammation, which plays a critical role in neurodegenerative disease.

LRP1 has been shown to be involved in the development of neurodegenerative diseases and in the regulation of the metabolism of amyloid-3 peptides (Aβs) in the brain and periphery. Neuroinflammation plays a critical role in neurodegenerative disease, such as Alzheimer's Disease (AD). LRP1 is highly expressed in the cells of the CNS and shown to play a critical role for the survival of primary neurons under stressful conditions.^{56,57} Microglial cells act as the resident immune cell of the brain, serving to maintain homeostasis in the environment. Microglia are considered the prototypic tissue-resident macrophage-like innate immune cells of the CNS.⁵⁸ These cells are involved in chemotaxis, phagocytosis, antigen presentation, and cytokine production, however, impaired or infected microglia can contribute to neuroinflammation and neurodegeneration.⁵⁹ Reactive glia cells (such as microglial cells) and associated neuroinflammation play a key role in both disease initiation and progression becoming activated through dysregulated clearance of beta amyloid and other damage associated molecular patterns (DAMPs)¹³ AB deposition and tau hyper-phosphorylation contribute to microglial activation, NFκB inflammatory pathway activation and associated pro-inflammatory cytokines such as TNFα, IL-6 and IL-1β, which contribute to neuronal damage and loss. Impaired autophagy, a homeostatic process that degrades and recycles proteins such as beta amyloid, has been associated with AD.⁷ LRP1 has been shown to mediate healthy lysosomal processing associated with autophagy. Therefore, through LRP1, the disclosed SERPIN peptide derivatives can mediate several aspects of AD including healthy cell metabolism to reduce the spread of protein aggregation, alleviate neuroinflammation and improve neuronal dysfunction leading to survival and possibly regeneration of these cells.

### Acute Lung Injury (Acute Respiratory Distress Syndrome)

Acute Lung Injury (ALI), leading to Acute Respiratory Distress Syndrome (ARDS), can be initiated by a variety of inflammatory insults such as pneumonia, traumatic injury, and/or infection. A key initiation step in ALI is the dysregulated innate immune response to damage associated or pathogen associated molecular patterns (DAMPs or PAMPs, respectively). Alveolar macrophages are activated by the infectious microbes in Toll-like and Nod-like Receptor signaling pathways that lead to further macrophage and circulating neutrophil recruitment. Neutrophils accumulate in the lungs and release proinflammatory cytokines and other cytotoxic substances causing exacerbation of the injury. The lung epithelium is damaged by these cells and their secreted products cause pulmonary edema and potential respiratory distress (ARDS). An increase in proinflammatory cytokines TNF-alpha, IL-1beta, IL-6, IL-8, and IL-18 occurs resulting in a “cytokine storm” which is predictive of morbidity and mortality in sepsis. LRP1 was reported to be a critical player in sepsis and ARDS as it regulates lung inflammation and lung tissue repair.⁴⁹ The SERPIN peptide derivatives of the present technology target LRP1, leading to the precise coordination of the immune response in order to restore homeostasis. Therefore, the disclosed peptide derivatives can have a significant impact to mitigate lung damage and improve survival through multiple mechanisms initiated by inducing specific LRP1-mediated signaling pathways. These mechanisms include rebalancing the cytokine output to promote inflammatory resolution, mediating autophagy to restore proper cell metabolic processes, restoring immune cell function and receptor scavenging to regulate the tissue injury cycle. These mechanisms help in infection clearance and allow the host to better fight infection. Based on these mechanisms, the disclosed SERPIN peptide derivatives can be used as immunomodulatory therapeutic agents to prevent or treat ARDS by mediating LRP1 signaling.

### Infectious Disease

Acute respiratory distress syndrome (ARDS) is a major complication in patients with severe COVID-19 illness. Clinical evaluation and retrospective studies out of Wuhan China show that all of the patients admitted to hospital presented with pneumonia, of those 29% developed ARDS and among critically ill patients who were non-survivors, a vast majority (81%) had developed ARDS.^{17,52} Therefore, patients who develop ARDS are at a substantially higher risk of death. Virally mediated activation of the innate immune response through the Toll-like Receptors (TLRs) initiates an inflammatory response that is necessary to eliminate the infection. In some cases, the initiating viral insults and triggers are highly amplified and uncontrolled, resulting in overstimulation of the immune cells and an aberrant cytokine release (known as a cytokine storm), resulting in tissue damage that can lead to organ failure and death. Cytokine regulation is a key factor in preventing the harmful effects of an overactive immune response and controlling the cytokine storm could have a significant impact on viral pneumonia progression to ARDS. In the case of SARS-CoV-2, emerging evidence suggests that for a subpopulation of patients with severe illness, the cytokine storm is a contributing factor to mortality.²⁷ Recently, clinical trials were launched for Kevzara (sarilumab), an interleukin-6 inhibitor (IL-6), for the treatment of coronavirus infected patients with lung complications. IL-6 is a biomarker associated with higher mortality rates in individuals with pneumonia. Those trials will provide critical insight into the effectiveness of single cytokine targeted therapeutics. However, historically, targeting a single cytokine or pathway in ARDS patients has not proved to be an effective approach.

LRP1 signaling mediated by the SERPIN peptide derivatives of the present technology may have therapeutic potential as an immunomodulatory strategy to improve COVID-19 patient outcomes through regulating several mechanisms of lung inflammation including curbing the cytokine storm, improving cell survival, regulating autophagy and cell metabolism to clear infection and controlling tissue repair signaling pathways to promote proper healing and prevent fibrosis. LRP1 is widely known to regulate protease/antiprotease activity and mediate viral entry.⁴³ The novel coronavirus utilizes proteases (TMPRSS2) to regulate cell entry and infection and treatment with its corresponding protease inhibitor such as SERPIN blocked lung cell infection.^{16,24,28} SARS-CoV-2 viral entry is dependent on a serine protease TMPRSS2 that primes the viral S protein involved in host cell entry, which is the first step in the viral replication cycle. Camostat mesylate is a natural SERPIN with protease inhibitor function of TMPRSS2 and was effective in reducing SARS-CoV-2 viral entry, which can limit both infection and spread of the disease.¹⁶ An alpha-1 antitrypsin derivative of the present technology such as SP163M is also capable of inhibiting TMPRSS2, potentially through LRP1 and may have effects in reducing SARS-CoV-2 viral replication. The disclosed SERPIN peptide derivatives may reduce SARS-CoV-2 replication via the mechanism of LRP1 control of the host protease/viral protein interaction to inhibit viral entry.

Arthropod-borne viruses are important causes of acute encephalitis and an emerging worldwide problem with an ever-growing risk for importation into new regions.^{11,31} The mosquito-borne encephalitic alphaviruses including Venezuelan (VEEV), eastern (EEEV) and western equine encephalitis viruses (WEEV) are endemic in the Americas and cause outbreaks of encephalomyelitis, which can spread into the United States. In humans VEEV causes a febrile illness typified by fever, malaise, and vomiting. In some cases, infection progresses to the central nervous system (CNS). Neurological cases have a mortality rate as high as 35% in children and 10% in adults, with long-term neurological deficits often seen in survivors.⁴² The overall estimate of VEEV survivors with neurological sequelae is 4-14% but may be even greater due to the misdiagnosis of arboviral encephalitis.³³

VEEV infection is known to inhibit cellular transcription and translation in order to downregulate the innate immune response.^{45,48} In contrast, in the central nervous system (CNS), VEEV infection results in the upregulation of numerous genes in the inflammatory response and apoptotic pathway.^{45,48} Specifically, pro-inflammatory cytokines including interleukin-1β (IL-1β), IL-6, IL-12, and tumor necrosis factor-α (TNF-α) play a role in VEEV pathogenesis.^{9,20,30,34,35} Gene expression changes were analyzed in the brain tissue of VEEV infected mice and alterations in immune pathways involved in antigen presentation, inflammation, apoptosis and the traditional antiviral response were discovered.³⁶ In addition, viral modulation of extracellular matrix and adhesion genes such as integrins, cadherin-1, cadherin-2, vascular cell adhesion molecule-1, and intracellular adhesion molecule-1 (ICAM-1) in the brains of VEEV infected mice was observed.³⁷ ICAM-1 knockout mice demonstrated reduced inflammation in the brain and a subsequent delay in the onset of clinical disease.³⁷ These studies suggest that alphavirus-induced inflammation contributes substantially to neurological damage and that control of inflammation is a viable therapeutic strategy.

The SERPIN peptide derivatives of the present technology can bind LRP1 and initiate the immune modulatory cascade. LRP1 expression increases during ischemia, tissue injury and viral infection.^{2,22,25,43} The binding of one or more disclosed peptide derivatives to LRP1 can inhibit the inflammatory response and induce pro-survival signaling through phosphorylation of protein kinase Akt. Therefore, targeting LRP1 has potential as a broad-spectrum therapeutic strategy for infectious disease.

Through LRP1 directed host mediated mechanisms, the SERPIN peptide derivatives can curb the harmful cytokine storm associated with severe COVID-19 disease, activate protective pathways to prevent lung damage, and/or clear infection faster and has direct impact on suppressing viral infection. Therefore, the dual anti-inflammatory and antiviral mechanism of the SERPIN peptide derivatives can have effects in improving survival in patients with SARS-COV2 induced ARDS.

### Eosinophilic Esophagitis

Eosinophilic Esophagitis (EoE) is a chronic, largely type-2 immune mediated allergic inflammatory response associated with esophageal dysfunction and disturbed epithelial barrier function. The esophageal inflammation results in trouble swallowing, persistent heartburn, chest and abdominal pain, weight loss and food impactions. It is characterized by a high number of eosinophils, proteases, cathelicidin, serine proteases including the kallikreins (KLK5), as well thymic stromal lymphopoietin (TSLP)—a cytokine and master regulator of allergic type-2 inflammatory responses in the local environment.⁴¹ In esophageal epithelial cells, a loss of the function of a serine peptidase inhibitor, Kazal type 7-SPINK7, results in uncontrolled protease activity, release of pro-inflammatory cytokines such as TNFα, CCL2, GM-CSF, IL-8, and CXCL10 and inflammation. Recently, it was found that the serine protease KLK-5, which is an important mediator of epithelial barrier function, is a direct target of SPINK7 and loss of SPINK7 mediates EoE pathogenesis largely through uncontrolled KLK-5 protease activity. Interestingly, the SERPIN Alpha-1 antitrypsin, is capable of inhibiting KLK5 activity in vitro and allergen-induced esophageal eosinophilia in vivo. However, the mechanism of the activity is not yet clear and may involve LRP1, instead of or in addition to direct proteolytic inhibition.

Patients with eosinophilic asthma have lower levels of LRP1. In addition, LRP1 deletion specifically of CD11b and CD11c dendritic cells in mice results in heightened allergic inflammatory response in an allergic airway disease model.²⁹ Mice with LRP1 deletion had increased antigen uptake and suffered increased eosinophilic inflammation, allergic sensitization, Th2 mediated cytokine production and a reduction in T-regulatory cells.²⁹ Therefore, LRP1 could aid in maintaining homeostasis of proteases/inhibitors in the esophageal environment, mediating the TH2 responses and inhibiting inflammatory signaling pathways (NFκB, JNK) resulting in repair of esophageal dysfunction.

As demonstrated herein, the disclosed SERPIN peptide derivatives such as SA7, a potent LRP1 agonist with no protease inhibitor function, can reduce TMPRSS2 expression. TMPRSS2 expression was shown to be significantly increased in the nasal and airway epithelial cells in type 2 asthma and allergic rhinitis. Studies have also shown that TMPRSS2 expression is positively associated with TH2 mediated immune responses important in allergic responses. The disclosed SERPIN peptide derivatives may have significant implications in alleviating eosinophilic esophagitis by mediating control over LRP1.

The following examples are intended to illustrate various embodiments of the present technology. As such, the specific embodiments discussed are not to be constructed as limitations on the scope of the present technology. It will be apparent to one skilled in the art that various equivalents, changes, and modifications may be made without departing from the scope of present technology, and it is understood that such equivalent embodiments are to be included herein. Further, all references cited in the disclosure are hereby incorporated by reference in their entirety, as if fully set forth herein.

Various embodiments of the present technology are set forth herein below.

Embodiment 1: A SERPIN peptide derivative comprising a core sequence of FVFLM (SEQ ID NO: 1), FVFL[Nle] (SEQ ID NO: 2), PFVFLM (SEQ ID NO: 8), PFVFL[Nle] (SEQ ID NO: 9), and one or more of the following modifications:
(i) a polar head added to the N-terminus of the core sequence, a polar tail added to the C-terminus of the core sequence, or both;
(ii) one or more amino acid residues added to the N-terminus of the core sequence, C-terminus of the core sequence, or both such that the peptide derivative can be cyclized;
(iii) one or more amino acid residues in the core sequence substituted by one or more substitute amino acid residues having less hydrophobicity compared to the one or more amino acid residues in the core sequence that is substituted;
(iv) one or more amino acid residues in the core sequence substituted by one or more substitute amino acid residues having greater hydrophobicity compared to the one or more amino acid residues in the core sequence that is substituted; and
(v) one or more amino acid residues in the core sequence are deleted.

Embodiment 2: The SERPIN peptide derivative of embodiment 1, wherein the SERPIN peptide derivative is a linear peptide.

Embodiment 3: The SERPIN peptide derivative of embodiment 1, wherein the SERPIN peptide derivative is a cyclized peptide.

Embodiment 4: The SERPIN peptide derivative of embodiment 3, wherein the SERPIN peptide derivative is cyclized by forming a disulfide bond between two Cys residues.

Embodiment 5: The SERPIN peptide derivative of embodiment 3, wherein the SERPIN peptide derivative is cyclized by a linker between two amino acid residues.

Embodiment 6: The SERPIN peptide derivative of any one of embodiments 1-5, wherein the polar head or the polar tail comprises two or more charged amino acids such as positively charged amino acids selected from the group consisting of Arg, Lys, and His.

Embodiment 7: The SERPIN peptide derivative of any one of embodiments 1-6, wherein the SERPIN peptide derivative has a size of less than 15 amino acid residues.

Embodiment 8: The SERPIN peptide derivative of any one of embodiments 1-7, wherein the SERPIN peptide derivative has a size of 7, 8, 9, 10, 11, or 12 amino acid residues.

Embodiment 9: The SERPIN peptide derivative of any one of embodiments 1-8, wherein one or more amino acid residues are D-amino acids.

Embodiment 10: A SERPIN peptide derivative comprising the amino acid sequence X1-X2-X3-X4-X5-X6-X7-X8-X9-X10-X11 (SEQ ID NO: 3), wherein:
X1 is a hydrophilic amino acid residue or none;
X2 is a hydrophilic amino acid residue or none;
X3 is a hydrophilic amino acid residue or none;
X4 is a Cys amino acid residue or none;
X5 is a Pro amino acid residue or none;
X6 is a first hydrophobic amino acid residue;
X7 is a short-branched amino acid residue;
X8 is a second hydrophobic amino acid residue;
X9 is a saturated hydrophobic amino acid residue;
X10 is a hydrophilic amino acid of D configuration; and
X11 is any amino acid residue which allows cyclization of the SERPIN peptide derivative.

Embodiment 11: The SERPIN peptide of embodiment 10, wherein the first and second hydrophobic amino acid residue are aromatic amino acid residues.

Embodiment 12: The SERPIN peptide of any of embodiments 10-11, wherein the short-branched amino acid residue is Val or Thr.

Embodiment 13: The SERPIN peptide of any of embodiments 10-12, wherein the saturated hydrophobic amino acid is Leu.

Embodiment 14: The SERPIN peptide of any of embodiments 10-13, wherein hydrophilic amino acid of D configuration is a D-configured Asp, Glu, Lys, Dap, or Cys residue.

Embodiment 15: The SERPIN peptide of embodiments 10-14, wherein
X1 is a basic residue displaying a positive charge or none;
X2 is a basic residue displaying a positive charge or none; and
X3 is a basic residue displaying a positive charge or none.

Embodiment 16: The SERPIN peptide derivative of embodiments 10-15, wherein
X1 is a Arg, Lys or His residue or none;
X2 is a Arg, Lys or His residue or none; and
X3 is a Arg, Lys or His residue or none.

Embodiment 17: The SERPIN peptide derivative of any one of embodiments 10-16, wherein X6 is Phe or Nal.

Embodiment 18: The SERPIN peptide derivative of any one of embodiments 10-17, wherein X8 is Phe or Nal.

Embodiment 19: The SERPIN peptide derivative of any one of embodiments 10-18, wherein X6 is Ala, Phe, or Nal, and X4 is Nal.

Embodiment 20: The SERPIN peptide derivative of any one of embodiments 10-19, wherein X7 is Asp, Glu, Lys, Dap, or Cys.

Embodiment 21: The SERPIN peptide derivative of any one of embodiments 10-20, wherein the SERPIN peptide derivative is linear or cyclized.

Embodiment 22: The SERPIN peptide derivative of any one of embodiments 10-21, wherein the SERPIN peptide derivative has a size of less than 15 amino acid residues.

Embodiment 23: The SERPIN peptide derivative of any one of embodiments 10-22, wherein the SERPIN peptide derivative has a size of 7, 8, 9, 10, 11, or 12 amino acid residues.

Embodiment 24: A SERPIN peptide derivative comprising a core sequence of FVFLM (SEQ ID NO: 1), FVFL[Nle] (SEQ ID NO: 2), PFVFLM (SEQ ID NO: 8), PFVFL[Nle] (SEQ ID NO: 9) and one or more of the following modifications:
(i) one or more Lys, Glu, or His residues added to the N-terminus of the core sequence, a polar tail added to the C-terminus of the core sequence, or both;
(ii) one or more amino acid residues added to the N-terminus of the core sequence, C-terminus of the core sequence, or both such that the SERPIN peptide derivative can be cyclized by forming a disulfide bond between two Cys residues;
(iii) one or more amino acid residues in the core sequence substituted by one or more Thr amino acid residues; and
(iv) one or more amino acid residues in the core sequence substituted by one or more substitute amino acid residues having greater hydrophobicity than the one or more amino acid residues that is substituted.

Embodiment 25: The SERPIN peptide derivative of embodiment 24, wherein the SERPIN peptide derivative is a linear peptide.

Embodiment 26: The SERPIN peptide derivative of embodiment 24 or 25, wherein the SERPIN peptide derivative has a size of less than 15 amino acid residues.

Embodiment 27: The SERPIN peptide derivative of any one of embodiments 24-26, wherein the SERPIN peptide derivative has a size of 7, 8, 9, 10, 11, or 12 amino acid residues.

Embodiment 28: A SERPIN peptide derivative comprising the amino acid sequence X1-X2-X3-X4-X5-X6-X7-X8-X9-X10-X11 (SEQ ID NO: 3), wherein:
X1 is a hydrophilic amino acid residue or none;
X2 is a hydrophilic amino acid residue or none;
X3 is a hydrophilic amino acid residue or none;
X4 is a Cys amino acid residue or none;
X5 is a Pro amino acid residue or none;
X6 is a first hydrophobic amino acid residue;
X7 is a short-branched amino acid residue;
X8 is a second hydrophobic amino acid residue;
X9 is a saturated hydrophobic amino acid residue;
X10 is a hydrophilic amino acid of D configuration; and
X11 is any amino acid residue which allows cyclization of the SERPIN peptide derivative;
wherein X4 is Cys, X5 is Pro, X7 is Thr, and/or X10 is a D-configured Lys residue.

Embodiment 29: The SERPIN peptide of embodiment 28, wherein the first and second hydrophobic amino acid residue are aromatic amino acid residues.

Embodiment 30: The SERPIN peptide of embodiment 28 or 29, wherein the short-branched amino acid residue is Val or Thr.

Embodiment 31: The SERPIN peptide of any of embodiments 28-30, wherein the saturated hydrophobic amino acid is Leu.

Embodiment 32: The SERPIN peptide of any of embodiments 28-31, wherein hydrophilic amino acid of D configuration is a D-configured Asp, Glu, Lys, Dap, or Cys residue.

Embodiment 33: The SERPIN peptide of any of embodiments 28-32, wherein
X1 is a basic residue displaying a positive charge or none;
X2 is a basic residue displaying a positive charge or none; and
X3 is a basic residue displaying a positive charge or none.

Embodiment 34: The SERPIN peptide derivative of any of embodiments 28-33, wherein
X1 is a Arg, Lys or His residue or none;
X2 is a Arg, Lys or His residue or none; and
X3 is a Arg, Lys or His residue or none.

Embodiment 35: The SERPIN peptide derivative of any one of embodiments 28-34, wherein X6 is Phe or Nal.

Embodiment 36: The SERPIN peptide derivative of any one of embodiments 28-35, wherein X8 is Phe or Nal.

Embodiment 37: The SERPIN peptide derivative of any one of embodiments 28-36, wherein X6 is Ala, Phe, or Nal, and X4 is Nal.

Embodiment 38: The SERPIN peptide derivative of any one of embodiments 28-37, wherein X7 is Asp, Glu, Lys, Dap, or Cys.

Embodiment 39: The SERPIN peptide derivative of any one of embodiments 28-38, wherein the SERPIN peptide derivative is linear or cyclized.

Embodiment 40: The SERPIN peptide derivative of any one of embodiments 28-39, wherein the SERPIN peptide derivative has a size of less than 15 amino acid residues.

Embodiment 41: The SERPIN peptide derivative of any one of embodiments 28-40, wherein the SERPIN peptide derivative has a size of 7, 8, 9, 10, 11, or 12 amino acid residues.

Embodiment 42: A SERPIN peptide derivative comprising a sequence of SEQ ID NOs: 10-20, or 23-62, wherein the SERPIN peptide derivative comprises a polar head added to the N-terminus of the core sequence, a polar tail added to the C-terminus of the core sequence, or both.

Embodiment 43: A SERPIN peptide derivative comprising a sequence of SEQ ID NOs: 15-17, or 23-62 wherein one or more amino acid residues added to the N-terminus of the core sequence, C-terminus of the core sequence, or both such that the peptide derivative can be cyclized.

Embodiment 44: A SERPIN peptide derivative comprising a sequence of SEQ ID NOs: 27-29, 38, 39, or 42-62, wherein one or more amino acid residues in the core sequence substituted by one or more substitute amino acid residues having less hydrophobicity compared to the one or more amino acid residues in the core sequence that is substituted.

Embodiment 45: A SERPIN peptide derivative comprising a sequence of SEQ ID NOs: 30 or 31, wherein one or more amino acid residues in the core sequence substituted by one or more substitute amino acid residues having greater hydrophobicity compared to the one or more amino acid residues in the core sequence that is substituted.

Embodiment 46: A SERPIN peptide derivative comprising a sequence of SEQ ID NOs: 41 or 47-53 wherein one or more amino acid residues in the core sequence are deleted.

Embodiment 47: A SERPIN peptide derivative comprising a sequence of SEQ ID NOs: 10, 11, 13, or 14, 35 or 36 wherein one or more Lys, Glu, or His residues added to the N-terminus of the core sequence, a polar tail added to the C-terminus of the core sequence, or both.

Embodiment 48: A SERPIN peptide derivative comprising a sequence of SEQ ID NOs: 15-17, or 23-31 wherein one or more amino acid residues added to the N-terminus of the core sequence, C-terminus of the core sequence, or both such that the SERPIN peptide derivative can be cyclized by forming a disulfide bond between two Cys residues.

Embodiment 49: A SERPIN peptide derivative comprising a sequence of SEQ ID NOs: 27-29, 38, 39, 47-55, or 59-62 wherein one or more amino acid residues in the core sequence substituted by one or more Thr amino acid residues.

Embodiment 50: A fusion protein comprising the SERPIN peptide derivative of any one of embodiments 1-49, and an epitope tag, a half-life extender, or both.

Embodiment 51: A conjugate comprising the SERPIN peptide derivative of any one of embodiments 1-49, and a permeability enhancer.

Embodiment 52: A pharmaceutical composition comprising the SERPIN peptide derivative of any one of embodiments 1-49, the fusion protein of embodiment 50, or the conjugate of embodiment 51.

Embodiment 53: The pharmaceutical composition of embodiment 52, further comprising one or more additional therapeutic agents.

Embodiment 54: The pharmaceutical composition of embodiment 52 or 53, further comprising a pharmaceutically acceptable carrier, excipient, additive, preservative, or a combination thereof.

Embodiment 55: The pharmaceutical composition of any one of embodiments 52-54, further comprising a permeability enhancer.

Embodiment 56: The pharmaceutical composition of any one of embodiments 52-55, wherein the pharmaceutical composition is formulated for oral administration, transdermal administration, or parenteral administration.

Embodiment 57: A fusion protein comprising the SERPIN peptide derivative of any one of embodiments 24-41, 45, or 47-49, and an epitope tag, a half-life extender, or both.

Embodiment 58: A conjugate comprising the SERPIN peptide derivative of any one of embodiments 24-41, 45, or 47-49, and a permeability enhancer.

Embodiment 59: A pharmaceutical composition comprising the SERPIN peptide derivative of any one of embodiments 24-41, 45, or 47-49, the fusion protein of embodiment 57, or the conjugate of embodiment 58.

Embodiment 60: The pharmaceutical composition of embodiment 59, further comprising one or more additional therapeutic agents.

Embodiment 61: The pharmaceutical composition of embodiment 59 or 60, further comprising a pharmaceutically acceptable carrier, excipient, additive, preservative, or a combination thereof.

Embodiment 62: The pharmaceutical composition of any one of embodiments 59-61, further comprising a permeability enhancer.

Embodiment 63: The pharmaceutical composition of any one of embodiments 59-62, wherein the pharmaceutical composition is formulated for oral administration, transdermal administration, or parenteral administration.

Embodiment 64: A method of treating a subject suffering from a disease or condition associated with LRP1, comprising administering to the subject an effective amount of the SERPIN peptide derivative of embodiments 1-49, the fusion protein of embodiment 50, the conjugate of embodiment 51, or the pharmaceutical composition of any of embodiments 52-56, to treat the disease or condition associated with LRP1.

Embodiment 65: The method of embodiment 64, wherein administering the SERPIN peptide reduces NFkB activation in the subject.

Embodiment 66: The method of embodiment 64 or 65, wherein administering the SERPIN peptide reduces TNFa in the subject.

Embodiment 67: The method of any of embodiments 64-66, wherein administering the SERPIN peptide reduces IL-6.

Embodiment 68: The method of any of embodiments 64-67, wherein the disease or condition associated with LRP1 is acute or neuropathic pain, nociceptive pain, or inflammatory pain.

Embodiment 69: The method of any of embodiments 64-68, wherein the disease or condition associated with LRP1 is a disease or condition caused by a viral infection.

Embodiment 70: The method of embodiment 69, wherein administering the SERPIN peptide reduces viral replication.

Embodiment 71: The method of any of embodiments 64-67, wherein the disease or condition associated with LRP1 is eosinophilic esophagitis.

Embodiment 72: The method of any of embodiments 64-67, wherein the disease or condition associated with LRP1 is acute lung injury.

Embodiment 73: A method of treating a subject suffering from a disease or condition associated with LRP1, comprising administering to the subject an effective amount of the SERPIN peptide derivative of any one of embodiments 24-41, 45, or 47-49, the fusion protein of embodiment 57, the conjugate of embodiment 58, or the pharmaceutical composition of any of embodiments 59-63 to treat the disease or condition associated with LRP1.

Embodiment 74: The method of embodiment 73, wherein administering the SERPIN peptide reduces NFkB activation in the subject.

Embodiment 75: The method of embodiment 73 or 74, wherein administering the SERPIN peptide reduces TNFa in the subject.

Embodiment 76: The method of any of embodiments 73-75, wherein administering the SERPIN peptide reduces IL-6.

Embodiment 77: The method of any of embodiments 73-75, wherein the disease or condition associated with LRP1 is a disease or condition caused by a viral infection.

Embodiment 78: The method of embodiment 77, wherein administering the SERPIN peptide reduces viral replication.

Embodiment 79: The method of any of embodiments 73-75, wherein the disease or condition associated with LRP1 is eosinophilic esophagitis.

Embodiment 80: The method of any of embodiments 73-75, wherein the disease or condition associated with LRP1 is acute lung injury.

### EXAMPLES

### Example 1: Anti-Inflammatory Effects of SERPIN-Derived Derivatives

SERPIN peptide derivatives SA1-SA8 listed in Table 1 were tested for their anti-inflammatory effects using SP163M and SP22 as positive controls. The reporter cells (THP1-XBlue-MD2-CD14 cells) were treated with each peptide derivative SA1-SA8 as well as SP22 and SP163M (50 μg/ml) before being insulted with LPS (5 ng/ml) and incubated overnight. The NFκB inducible Secreted Embryonic Alkaline Phosphatase (SEAP) was measured in the supernatant and read for absorbance. As shown in FIG. 1, the SERPIN peptide derivatives that retain the shortest LRP1 binding sequence FVFL[Nle] and an RRR tripeptide as a flanker to improve solubility (such as SA3, SA6, and SA7) demonstrated improved activity in NFκB inhibition when compared to SP163M. However, the SERPIN peptide derivatives flanked with either HHH or KKK tripeptide or with negatively charged EEE tripeptide (such as SA1, SA2, SA4, SA5) demonstrated minimal NFκB inhibition activity.

This data shows that a shortened peptide (e.g., SA3) can be made and that the shortened peptide is more effective in reducing NFκB activation than the SERPIN peptides SP163M and SP22, which contain the highly conserved core sequence including FNKP and LRP1 binding motifs (FVFLM/Nle).

### Example 2: Assessment of Biological Activities of SERPIN Peptide Derivatives

Various SERPIN peptide derivatives were screened in vitro for their ability to reduce TNFα and NFκB secretion via LRP-1 activation. The NFκB reporter cells (THP1-XBlue-MD2-CD14 cells) were treated with each peptide derivative at various concentrations, up to 100 μg/ml before being insulted with LPS (5 ng/ml) and incubated overnight. The NFκB inducible Secreted Embryonic Alkaline Phosphatase (SEAP) was measured in the supernatant and read for absorbance. The percentage of reduction in NFκB activation vs. vehicle (LPS stimulated) cells is shown in the figures. IMG Microglial cells were treated with each peptide derivative at various concentrations, up to 100 μg/ml before LPS (100 ng/ml) stimulation for 24 hours. An ELISA was used to measure TNFα in the supernatant (μg/ml).

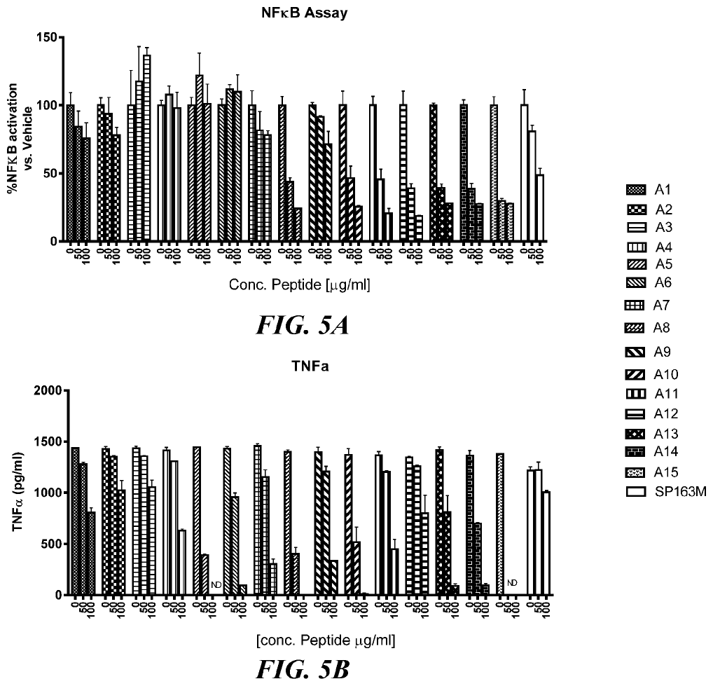

As shown in FIG. 5, SERPIN peptide derivatives A1-A15 listed in Tables 2-4 were tested for their TNFα activities. Peptide derivatives A5 and A8 having amino acid substitutions, as well as peptide derivatives A10 and A15 having modified ring closure demonstrated improved activities in reducing TNFα activation.

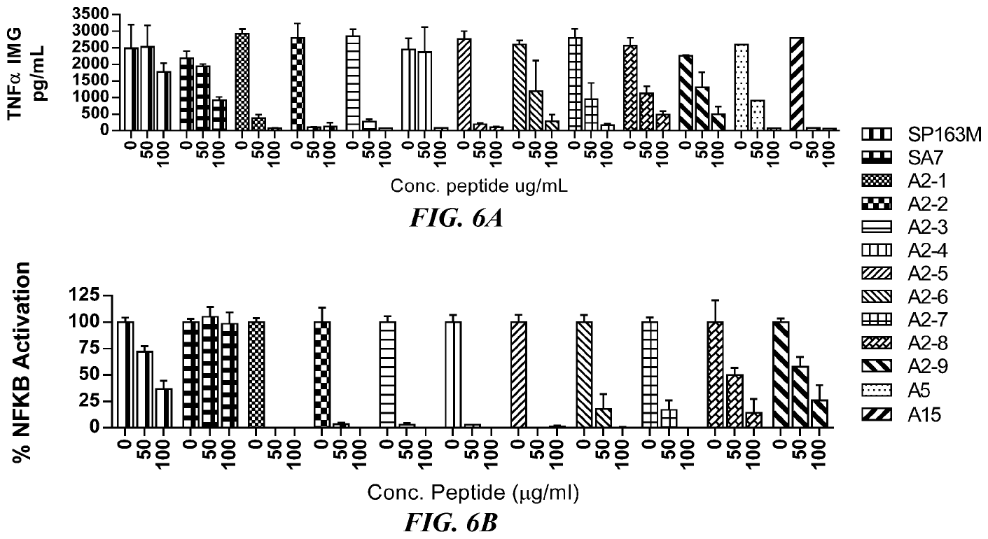

FIG. 6 demonstrates that peptide derivatives A2-1, A2-2, A2-3, A2-4, and A2-5 exhibited superior activities in reducing TNFα (FIG. 6A) and NFκB (FIG. 6B) activation. The activities of these peptide derivatives were dose-dependent and showed improved potency (FIG. 7). The peptide derivatives were tested at concentrations up to 50 μg/mL, while SP16 unmodified, SP163M and SA7 were tested at concentrations up to 100 μg/mL. None of the peptide derivatives show cytotoxicity (FIG. 8). For the cytotoxic assay, the cells (NFκB reporter cells or IMG microglial cells) were treated with either SP163M or peptide derivatives SA7, A5, A15, A2-1, A2-2, A2-3, A2-4, A2-5, A2-6, A2-7, A2-8, and A2-9 at concentrations up to 100 μg/mL for 24 hours and then cell viability was determined using the CellTiter-Glo® Luminescent Cell Viability Assay according to manufacturer's instruction.

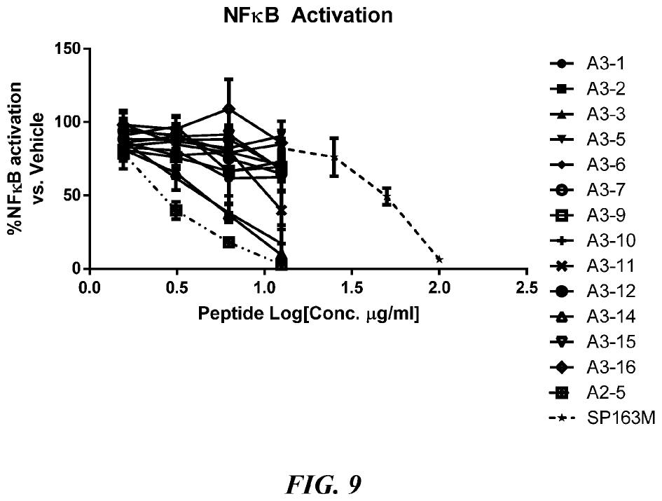

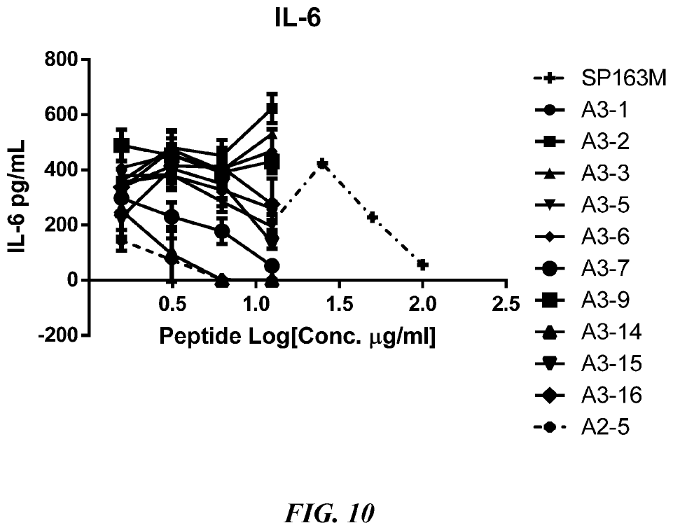

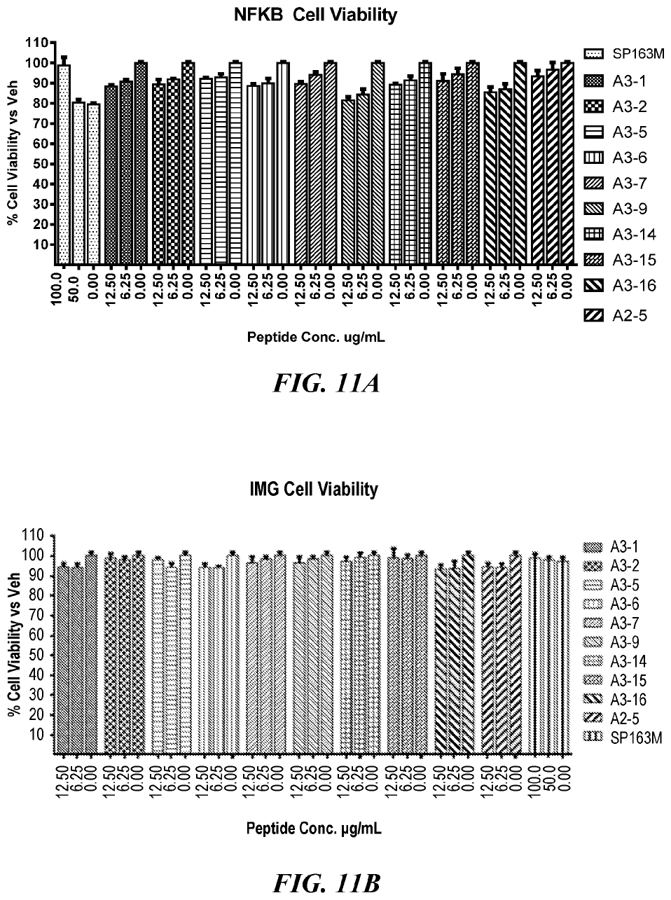

FIG. 9 demonstrates that peptide derivatives A3-10 and A3-14 exhibited superior activities in reducing NFκB activation, and FIG. 10 demonstrates that peptide derivative A3-14 exhibited superior activities in reducing IL-6 secretion in IMG after LPS stimulation. Peptide derivative A3-10 was not included in the IL-6 assay due to issues related to synthesis. None of these peptide derivatives tested showed any significant cytotoxicity (FIG. 11).

Table 7 below summarizes the EC50 of the in vitro test for some of the peptide derivatives of the present technology.

TABLE 7
EC50 of Various Peptide Derivatives (μg/mL)

| Peptide | NFκB | TNFα | IL-6 |
|---|---|---|---|
| A15 | 5.053 | 36.75 | 32.39 |
| A2-1 | 5.77 | 12.99 | 9.644 |
| A2-3 | 2.647 | 11.76 | 3.379 |
| A2-4 | 5.247 | 12.54 | 11.27 |
| A2-5 | 1.465 | 2.386 | 1.399 |
| A3-7 | 22.41 | n/a | 3.651 |
| A3-10 | 2.162 | 34.59 | 58.46 |
| A3-14 | 3.775 | 6 | 0.605 |
| SP163M | 41.51 | 125.9 | 134.5 |
| SP16 | 151.9 | ~617.9 | n/a |
| SA7 | 470 | 124.3 | 318.5 |

### Example 3: In Vivo Effects of SERPIN Peptide Derivatives

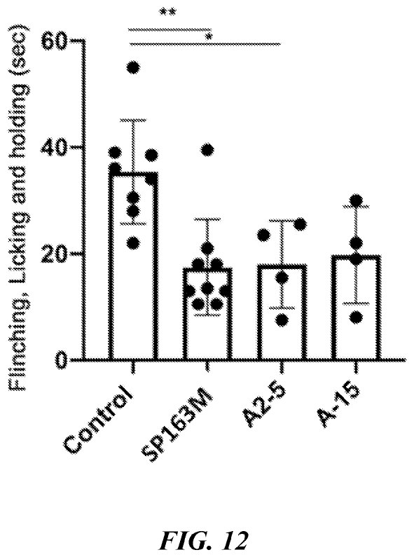

SERPIN peptide derivatives A15 and A2-5 were tested for their ability to diminish pain related behaviors induced by capsaicin in mice in comparison to SP163M. SP163M (50 μg), A15 (5 μg) or A2.5 (5 μg) or Capsaicin (25 ng) was injected into the hind paw in male mice and observed for 10 minutes for pain related behaviors such as flinching or licking. As shown in FIG. 12, both peptide derivatives at a low dose of 5 μg/mouse exhibited a level of pain blocking effect similar to that of SP163M at a much higher dose.

### Example 4: Effects of SERPIN Peptide Derivatives on Neuroinflammation

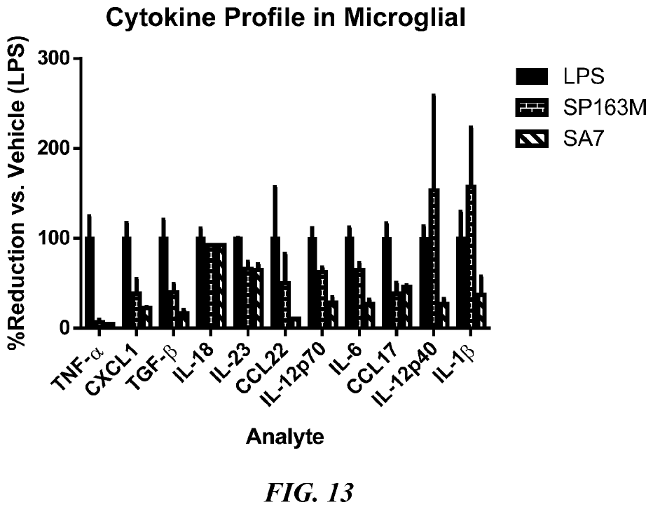

As shown in FIG. 13, the cytokine profile was explored in LPS induced microglial cells treated with SERPIN peptide derivative SA7, SP163M or vehicle control. Cytokines were induced with LPS and measured in the supernatant. In microglial cells, SA7 reduced several LPS mediated cytokines including IL-6, TNFα, IL-13, IL-12 and CXCL1 more potently than peptide SP163M. The IMG microglial cells were treated with SA7 (100 μg/ml), SP163M (100 μg/ml), 24 hours with and without LPS (100 ng/ml). Cytokines levels (μg/ml) in the supernatant were measured by multiplex assay.

Next, the efficacy of the peptide derivative SA7 was explored in an in vivo model of neuroinflammation. The LPS model of neuroinflammation has been used in numerous studies to understand neurodegenerative disease.⁶⁰⁻⁶³ These studies confirm that in C57BL/6 mice, LPS causes cognitive impairment. Further, acute systemic LPS causes activation of microglial cells in the brain, impaired amyloid beta clearance and an increase in blood and brain pro-inflammatory cytokines. Therefore, the LPS induced neuroinflammatory model was used to test the activity of the peptide derivative SA7.

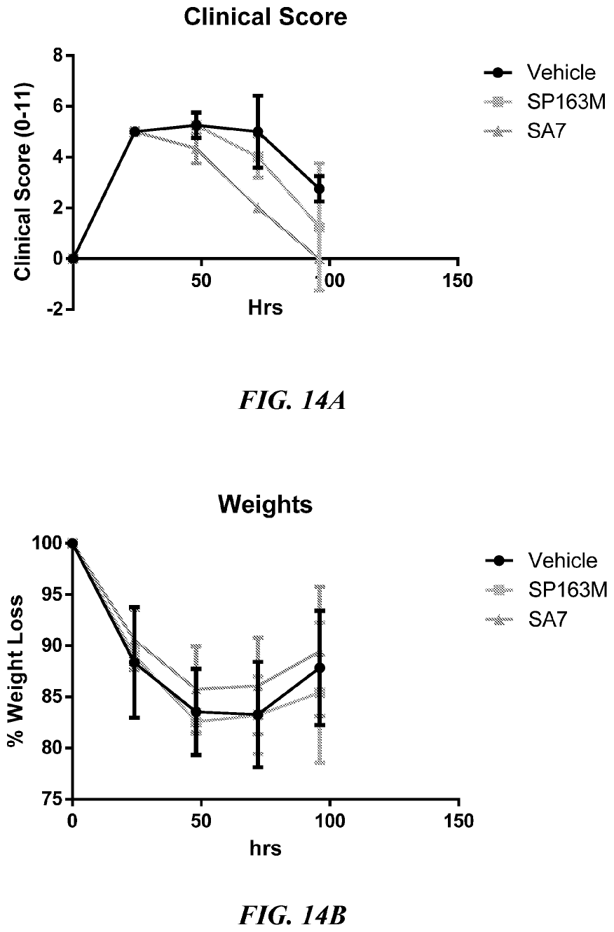

Female C57BL/6 mice (n=3-4) were administered with 1 mg/kg LPS (E. coli 0111: B4) by intraperitoneal injection for 4 consecutive days followed by either SP163M (100 μg) or SA7 (100 μg) administered subcutaneously daily, 1 hour following the LPS injection. Weights and clinical assessments were recorded daily. At the end of the study (Day 5) (24 hours after final LPS injection), the mice were weighed, scored and sacrificed. FIG. 3 shows that the clinical scores of SA7 treated mice were significantly improved compared to both vehicle and SP163M treated mice (FIG. 14A). There was also a slight benefit in weight loss with SA7 treated mice not seen with the SP163M treated mice (FIG. 14B).

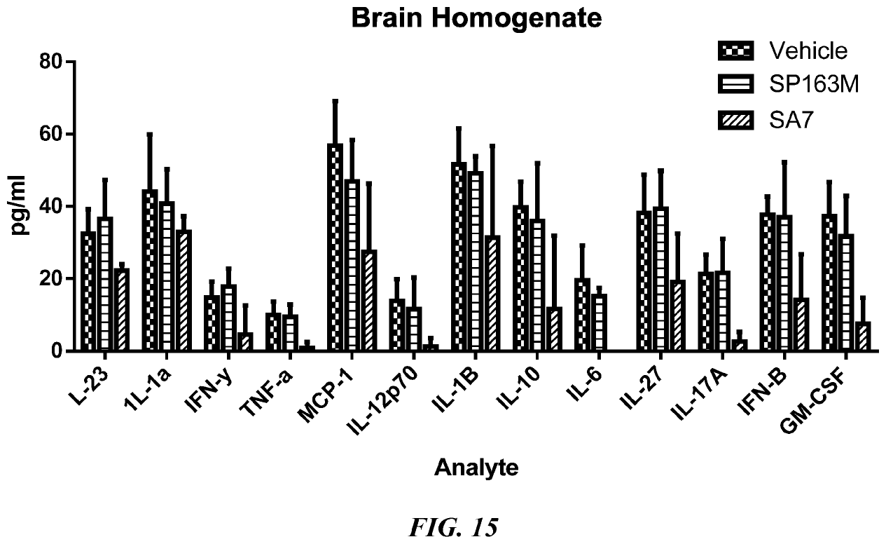

Brain homogenate was assessed for cytokine analysis using the LEGENDplex™ Mouse Inflammation Panel, a flow based multiplex assay using fluorescence-encoded beads. This panel allows simultaneous quantification of 13 mouse cytokines, including IL-1α, IL-1β, IL-6, IL-10, IL-12p70, IL-17A, IL-23, IL-27, MCP-1, IFN-β, IFN-γ, TNF-α, and GM-CSF. FIG. 15 shows that compared to vehicle treated and SP163M treated animals, SA7 treated group did show differences in the levels of many of the LPS induced cytokines. For instance, moderate levels of IL-6 were measured in the brain homogenate of vehicle and SP163M, but no detectable levels of IL-6 were measured in any of the SA7 treated animals, while IL-17A, IL-12, TNFα, and GM-CSF, also showed significantly lower levels. This corresponds to the improvement in clinical signs that were seen with SA7 treatment.

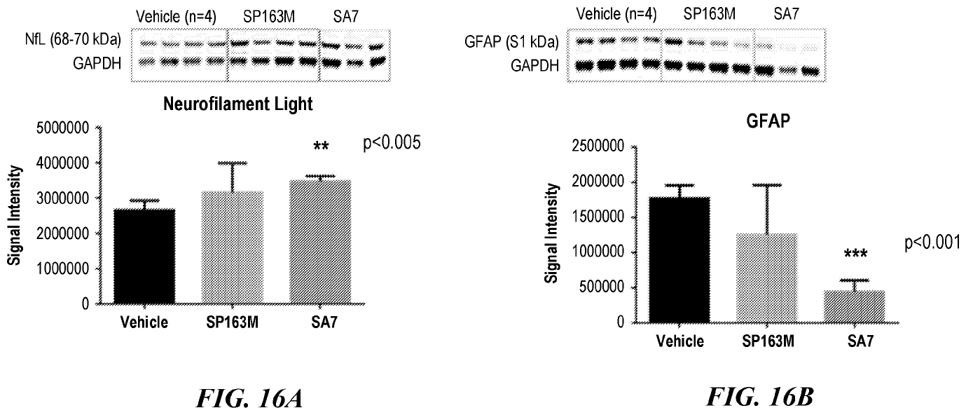

Next, the brain homogenate was analyzed for two biomarkers for CNS injury, neurofilament light chain (NfL) expressed in neuronal axons and glial fibrillary acidic protein (GFAP) expressed in astrocytes, a marker of astrocyte activation. There have been reports of inverse correlation between levels of inflammation in cortical areas and plasma NfL levels. LPS was found to selectively decrease neurofilament light chain expression in neurons. NfL is required for neuronal regeneration, synaptic connections, and the homeostatic signaling operations of neurons, indicating that an increase of NfL may indicate protection from LPS induced damage of neurons. FIG. 16A shows that peptide derivative SA7 significantly increases NfL protein expression in the brain homogenate following LPS activated neuroinflammation, compared to both vehicle and SP163M, as measured by western blot. Abnormal expression of GFAP is indicative of reactive astrocytes and neuroinflammation. FIG. 16B shows that peptide derivative SA7 significantly decreases GFAP expression in the brain lysate as measured by western blot analysis compared to both SP163M and vehicle treated animals. These results suggest a highly neuroprotective effect of the peptide derivatives.

### Example 5: Effects of SERPIN Peptide Derivatives on Autophagy

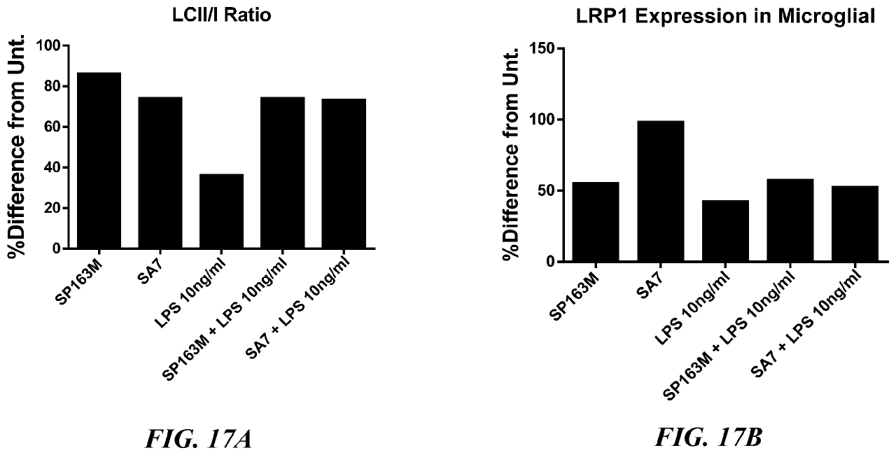

In addition to neuroinflammation, impaired autophagy, a homeostatic process that degrades and recycles proteins such as beta amyloid, has been associated with AD.⁷ LRP1 has been shown to mediate healthy lysosomal processing associated with autophagy. Therefore, through LRP1 agonist such as derivatives derived from SERPIN peptides have the potential to mediate several aspects of AD including healthy cell metabolism to reduce the spread of protein aggregation, alleviate neuroinflammation and improve neuronal dysfunction leading to survival and possibly regeneration of these cells. The effects of SP163M and peptide derivative SA7 on autophagy markers were tested in microglial cells. IMG microglial cells were treated with SP163M (100 μg/ml) or SA7 (100 μg/ml) before addition of LPS (100 ng/ml) for 24 hours. Lysates were collected and western blot analysis of both LRP1 and microtubule-associated protein light chain 3 (LC3 I and II) was performed. The LC3II/I ratio, a commonly used marker for autophagy indicating autophagic flux, was determined. FIG. 17A shows that in LPS activated microglial cells, the autophagic flux was reduced, indicating impairment of normal lysosomal processing. Treatment with either SP163M or peptide derivative SA7 in the presence of LPS restored autophagic functioning to near baseline levels. LRP1 protein expression was measured and FIG. 17B shows that LPS caused decreased levels (perhaps an indication of increased LRP1 shedding). SP163M and peptide derivative SA7 increased LRP1 protein expression in LPS activated microglial cells. Interestingly, in the absence of LPS, treatment with SA7 significantly increased LRP1 expression compared to SP163M. These results indicate another mechanism by which SP163M regulation of LRP1 contributes to homeostatic balance of dysregulated cell processes.

### Example 6: Effects of SERPIN Peptide Derivatives on Viral Infection

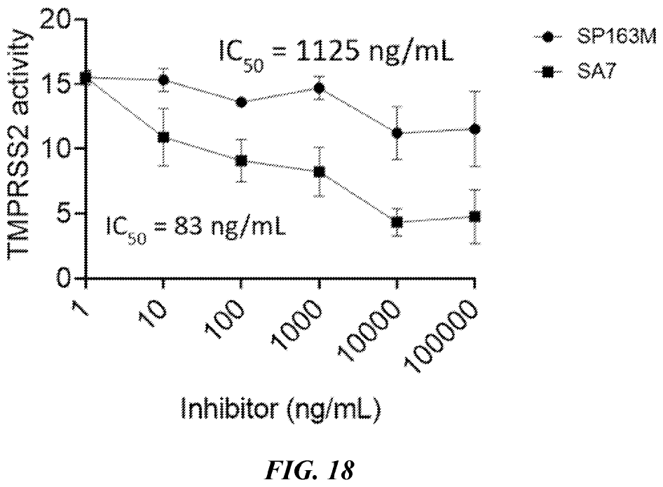

SERPIN-derived peptide SP163M demonstrated anti-viral effects. SP163M was able to significantly reduce viral replication of neuroinflammatory alphaviruses such as Eastern Equine Encephalitis (EEEV). SP163M also suppressed viral replication of the novel coronavirus, SARS-CoV-2. It is known that SARS-CoV-2 as well as other viruses such as influenza use host cell proteases for viral entry. TMPRSS2 processes the S protein on the SARS-CoV2 envelope in a process called priming. Priming of the S protein is necessary for binding between the S protein and the host receptor ACE2. LRP1 is widely known to regulate protease/antiprotease activity. Protease inhibitors, such as alpha-1 antitrypsin reduce proteolytic activity of TMPRSS2 preventing the priming of the S protein and therefore block virus entry. In addition, inhibition of TMPRSS2 prevents processing of ACE2, which decreases the infectivity of the coronavirus. FIG. 18 shows the inhibitory effect of SP163M and peptide derivative SA7 against TMPRSS2 at various concentrations. The IC50 of SP163M was 1125 ng/mL, while the SA7 derivative was much more potent with an IC50 of 83 ng/mL.

### Example 7: Effects of SERPIN Peptide Derivatives on Eosinophilic Esophagitis

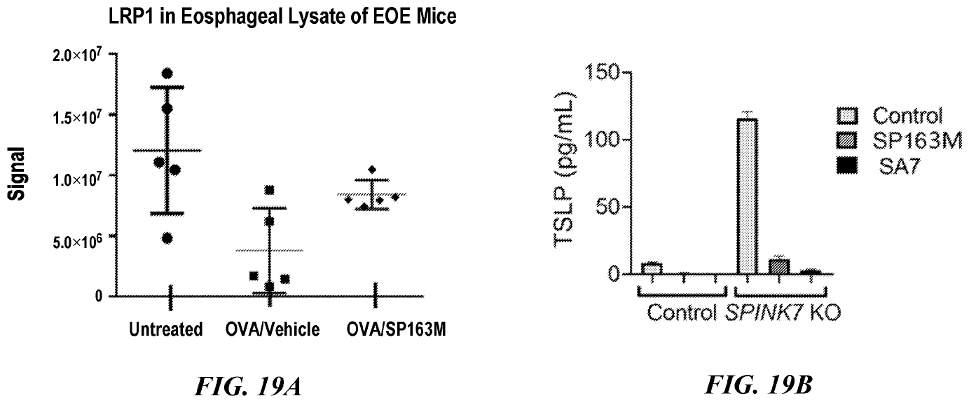

In terms of Eosinophilic Esophagitis (EoE), A1AT has been shown to attenuate experimental EoE in a murine model and in vitro. It is unknown whether the A1AT effects are mediated by inhibition of proteolytic activity or through activating LRP1 signaling. Patients with eosinophilic asthma have lower LRP1 and in animal models, loss of LRP1 is associated with worsened allergic responses. SP163M does not contain any sequences for anti-protease activity and is likely working through mediation of LRP1 but not through proteolytic activity. In a mouse model of Eosinophilic Esophagitis in which mice were sensitized intraperitoneally to ovalbumin (OVA) and then challenged intranasally on 4 separate days with OVA, the esophageal lysate of EoE induced mice show lower levels of LRP1 protein expression as measured by western blot analysis. Treatment of the mice with SERPIN-derived peptide SP163M shows increased levels of LRP1 (FIG. 19A). Evidence shows that loss of esophageal expression of the serine peptidase inhibitor, kazal type 7 (SPINK7) is an upstream event in EoE pathogenesis. Therefore, the in vitro effects of SP163M and peptide derivative SA7 were tested in reducing thymic stromal lymphopoietin (TSLP), a key immune checkpoint cytokine involved in stimulating dendritic cells to polarize the generation of type 2 T cells (Th2), key cells involved in EoE pathogenesis, following loss of SPINK7. FIG. 19B shows that SP163M inhibited polyI:C-induced TSLP production by SPINK7 knockout EPC2 cells (Human esophageal epithelial cells). SPINK7 KO cells and control cells were plated in high calcium and high density for 48 hours before being treated with either SP163M or derivative SA7 (200 μg/ml) and Poly I:C (5 μg/ml, or untreated) for 8 hours. TSLP production in the supernatant was measured by ELISA.

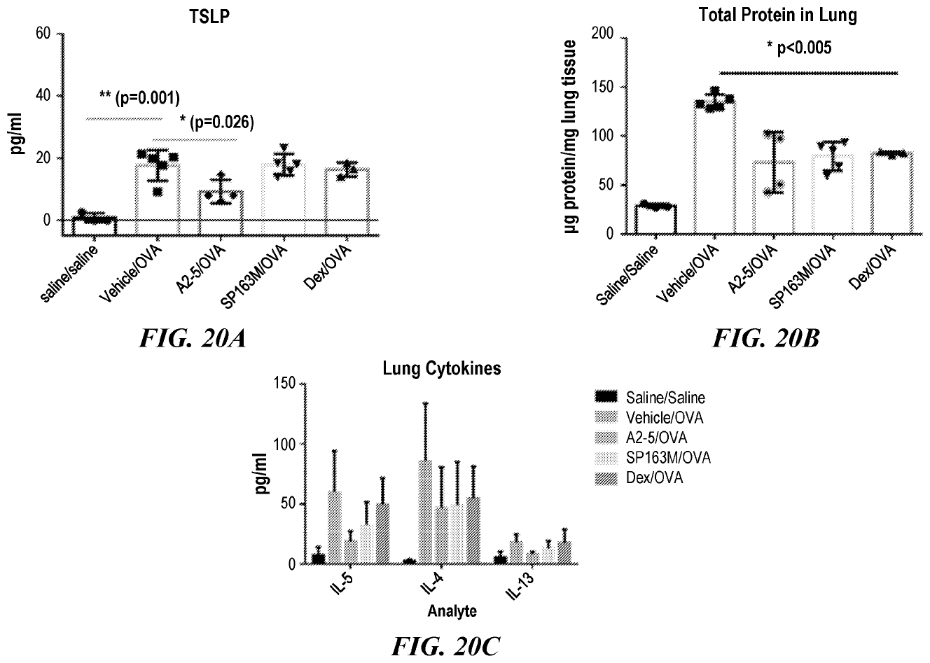

Further in an allergic asthma mouse model in which the mice were sensitized to ovalbumin by two intraperitoneal injections containing the adjuvant alum, a strong inducer of both innate and TH2 mediated immune responses and then challenged with ovalbumin given by intranasal instillation on four separate days, the mice treated with peptide derivative A2-5 showed a reduction in inflammatory mediators. Lung homogenate of ova-induced mice treated with vehicle (vehicle/OVA) demonstrated significantly increased levels of TSLP compared to non-ova induced mice (saline/saline) (p=0.001). Ova-induced mice treated with peptide derivative A2-5 (A2-5/OVA) demonstrated significantly lower levels of TSLP compared to ova-induced vehicle treated mice (p=0.026) (FIG. 20A). Allergen exposure also resulted in an increase in the total protein in the lung tissue compared to non-exposed animals. Infiltration of immune cells into the lung likely account for the increase in total protein levels in the lung. FIG. 20B shows that protein levels in the lung of mice treated with SP163M (SP163M/OVA), the steroid dexamethasone (Dex/OVA) and peptide derivative A2-5 significantly decreased compared to vehicle treated animals (p<0.005). In A2-5 treated mice, this was associated with a reduction of TH-2 mediated cytokines IL-5, IL-4 and IL-13 in the lung tissue compared with vehicle treated mice (FIG. 20C).

### REFERENCES

The references, patents and published patent applications listed below, and all references cited in the specification above are hereby incorporated by reference in their entirety, as if fully set forth herein
1. Berta, T., Y. C. Liu, Z. Z. Xu and R. R. Ji (2013). “Tissue plasminogen activator contributes to morphine tolerance and induces mechanical allodynia via astrocytic IL-1beta and ERK signaling in the spinal cord of mice.” Neuroscience 247: 376-385.
2. Christia, P., M. Bujak, C. Gonzalez-Quesada, W. Chen, M. Dobaczewski, A. Reddy and N. G. Frangogiannis (2013). “Systematic characterization of myocardial inflammation, repair, and remodeling in a mouse model of reperfused myocardial infarction.” J Histochem Cytochem 61(8): 555-570.
3. Cooke, J. P. (2019). “Inflammation and Its Role in Regeneration and Repair.” Circ Res 124(8): 1166-1168.
4. Feng, Y., B. Liu, X. Zheng, L. Chen, W. Chen and Z. Fang (2019). “The protective role of autophagy in sepsis.” Microb Pathoq 131: 106-111.
5. Franchini, M. and M. Montagnana (2011). “Low-density lipoprotein receptor-related protein 1: new functions for an old molecule.” Clin Chem Lab Med 49(6): 967-970.
6. Fregnan, F., L. Muratori, A. R. Simões, M. G. Giacobini-Robecchi and S. Raimondo (2012). “Role of inflammatory cytokines in peripheral nerve injury.” Neural regeneration research 7(29): 2259-2266.
7. Gali, C. C., E. Fanaee-Danesh, M. Zandl-Lang, N. M. Albrecher, C. Tam-Amersdorfer, A. Stracke, V. Sachdev, F. Reichmann, Y. Sun, A. Avdili, M. Reiter, D. Kratky, P. Holzer, A. Lass, K. K. Kandimalla and U. Panzenboeck (2019). “Amyloid-beta impairs insulin signaling by accelerating autophagy-lysosomal degradation of LRP-1 and IR-β in blood-brain barrier endothelial cells in vitro and in 3×Tg-AD mice.” Molecular and Cellular Neuroscience 99: 103390.
8. Gettins, P. G. W. and S. T. Olson (2016). “Inhibitory serpins. New insights into their folding, polymerization, regulation and clearance.” The Biochemical journal 473(15): 2273-2293.
9. Griffin, D., B. Levine, W. Tyor, S. Ubol and P. Despres (1997). “The role of antibody in recovery from alphavirus encephalitis.” Immunol Rev 159: 155-161.
10. Grosso, R. A., P. V. Subirada Caldarone, M. C. Sanchez, G. A. Chiabrando, M. I. Colombo and C. M. Fader (2018). “Hemin induces autophagy in a leukemic erythroblast cell line through the LRP1 receptor.” Biosci Rep.
11. Gubler, D. J. (2002). “The global emergence/resurgence of arboviral diseases as public health problems.” Archives of Medical Research 33(4): 330-342.
12. Hashizume, H., J. A. DeLeo, R. W. Colburn and J. N. Weinstein (2000). “Spinal glial activation and cytokine expression after lumbar root injury in the rat.” Spine (Phila Pa 1976) 25(10): 1206-1217.
13. Hemonnot, A.-L., J. Hua, L. Ulmann and H. Hirbec (2019). “Microglia in Alzheimer Disease: Well-Known Targets and New Opportunities.” Frontiers in Aging Neuroscience 11(233).
14. Herz, J. and D. K. Strickland (2001). “LRP: a multifunctional scavenger and signaling receptor.” J Clin Invest 108(6): 779-784.
15. Hoffmann, M., H. Kleine-Weber, S. Schroeder, N. Kruger, T. Herrler, S. Erichsen, T. S. Schiergens, G. Herrler, N.-H. Wu, A. Nitsche, M. A. Müller, C. Drosten and S. Pöhlmann (2020). “SARS-CoV-2 Cell Entry Depends on ACE2 and TMPRSS2 and Is Blocked by a Clinically Proven Protease Inhibitor.” Cell.
16. Hoffmann, M., H. Kleine-Weber, S. Schroeder, N. Kruger, T. Herrler, S. Erichsen, T. S. Schiergens, G. Herrler, N. H. Wu, A. Nitsche, M. A. Müller, C. Drosten and S. Pöhlmann (2020). “SARS-CoV-2 Cell Entry Depends on ACE2 and TMPRSS2 and Is Blocked by a Clinically Proven Protease Inhibitor.” Cell 181(2): 271-280.e278.
17. Huang, C., Y. Wang, X. Li, L. Ren, J. Zhao, Y. Hu, L. Zhang, G. Fan, J. Xu, X. Gu, Z. Cheng, T. Yu, J. Xia, Y. Wei, W. Wu, X. Xie, W. Yin, H. Li, M. Liu, Y. Xiao, H. Gao, L. Guo, J. Xie, G. Wang, R. Jiang, Z. Gao, Q. Jin, J. Wang and B. Cao (2020). “Clinical features of patients infected with 2019 novel coronavirus in Wuhan, China.” The Lancet 395(10223): 497-506.
18. Joslin, G., R. J. Fallon, J. Bullock, S. P. Adams and D. H. Perlmutter (1991). “The SEC receptor recognizes a pentapeptide neodomain of alpha 1-antitrypsin-protease complexes.” J Biol Chem 266(17): 11282-11288.
19. Kawamura, A., D. Baitsch, R. Telgmann, R. Feuerborn, G. Weissen-Plenz, C. Hagedorn, K. Saku, S. M. Brand-Herrmann, A. von Eckardstein, G. Assmann and J. R. Nofer (2007). “Apolipoprotein E interrupts interleukin-1 beta signaling in vascular smooth muscle cells.” Arterioscler Thromb Vasc Biol 27(7): 1610-1617.
20. Kehn-Hall, K., A. Narayanan, L. Lundberg, G. Sampey, C. Pinkham, I. Guendel, R. Van Duyne, S. Senina, K. L. Schultz, E. Stavale, M. J. Aman, C. Bailey and F. Kashanchi (2012). “Modulation of GSK-3beta activity in Venezuelan equine encephalitis virus infection.” PLoS One 7(4): e34761.
21. Landowski, L. M., M. Pavez, L. S. Brown, R. Gasperini, B. V. Taylor, A. K. West and L. Foa (2016). “Low-density Lipoprotein Receptor-related Proteins in a Novel Mechanism of Axon Guidance and Peripheral Nerve Regeneration.” The Journal of biological chemistry 291(3): 1092-1102.
22. Lillis, A. P., L. B. Van Duyn, J. E. Murphy-Ullrich and D. K. Strickland (2008). “LDL receptor-related protein 1: unique tissue-specific functions revealed by selective gene knockout studies.” Physiol Rev 88(3): 887-918.
23. Mantuano, E., M. S. Lam, M. Shibayama, W. M. Campana and S. L. Gonias (2015). “The NMDA receptor functions independently and as an LRP1 co-receptor to promote Schwann cell survival and migration.” Journal of Cell Science 128(18): 3478-3488.
24. Matsuyama, S., N. Nao, K. Shirato, M. Kawase, S. Saito, I. Takayama, N. Nagata, T. Sekizuka, H. Katoh, F. Kato, M. Sakata, M. Tahara, S. Kutsuna, N. Ohmagari, M. Kuroda, T. Suzuki, T. Kageyama and M. Takeda (2020). “Enhanced isolation of SARS-CoV-2 by TMPRSS2-expressing cells.” Proceedings of the National Academy of Sciences 117(13): 7001.
25. May, P. (2013). “The low-density lipoprotein receptor-related protein 1 in inflammation.” Curr Opin Lipidol 24(2): 134-137.
26. May, P., H. H. Bock and J. R. Nofer (2013). “Low density receptor-related protein 1 (LRP1) promotes anti-inflammatory phenotype in murine macrophages.” Cell Tissue Res 354(3): 887-889.
27. Mehta, P., D. F. McAuley, M. Brown, E. Sanchez, R. S. Tattersall and J. J. Manson (2020). “COVID-19: consider cytokine storm syndromes and immunosuppression.” The Lancet 395(10229): 1033-1034.
28. Meyer, M. and I. Jaspers (2015). “Respiratory protease/antiprotease balance determines susceptibility to viral infection and can be modified by nutritional antioxidants.” American journal of Physiology. Lung cellular and molecular physiology 308(12): L1189-L1201.
29. Mishra, A., X. Yao, A. Saxena, E. M. Gordon, M. Kaler, R. A. Cuento, A. V. Barochia, P. K. Dagur, J. P. McCoy, K. J. Keeran, K. R. Jeffries, X. Qu, Z.-X. Yu and S. J. Levine (2018). “Low-density lipoprotein receptor&#x2013; related protein 1 attenuates house dust mite&#x2013; induced eosinophilic airway inflammation by suppressing dendritic cell&#x2013; mediated adaptive immune responses.” Journal of Allergy and Clinical Immunology 142(4): 1066-1079.e1066.
30. Muehlenbein, M. P., F. B. Cogswell, M. A. James, J. Koterski and G. V. Ludwig (2006). “Testosterone correlates with Venezuelan equine encephalitis virus infection in macaques.” Virol J 3: 19.
31. Olival, K. J. and P. Daszak (2005). “The ecology of emerging neurotropic viruses.” Journal of Neurovirology 11(5): 441-446.
32. Rauch, J. N., G. Luna, E. Guzman, M. Audouard, C. Challis, Y. E. Sibih, C. Leshuk, I. Hernandez, S. Wegmann, B. T. Hyman, V. Gradinaru, M. Kampmann and K. S. Kosik (2020). “LRP1 is a master regulator of tau uptake and spread.” Nature 580(7803): 381-385.
33. Ronca, S. E., K. T. Dineley and S. Paessler (2016). “Neurological Sequelae Resulting from Encephalitic Alphavirus Infection.” Front Microbiol 7: 959.
34. Schoneboom, B. A., K. M. Catlin, A. M. Marty and F. B. Grieder (2000). “Inflammation is a component of neurodegeneration in response to Venezuelan equine encephalitis virus infection in mice.” J Neuroimmunol 109(2): 132-146.
35. Schoneboom, B. A., J. S. Lee and F. B. Grieder (2000). “Early expression of IFN-alpha/beta and iNOS in the brains of Venezuelan equine encephalitis virus-infected mice.” J Interferon Cytokine Res 20(2): 205-215.
36. Sharma, A., B. Bhattacharya, R. K. Puri and R. K. Maheshwari (2008). “Venezuelan equine encephalitis virus infection causes modulation of inflammatory and immune response genes in mouse brain.” BMC Genomics 9: 289.
37. Sharma, A., M. Bhomia, S. P. Honnold and R. K. Maheshwari (2011). “Role of adhesion molecules and inflammation in Venezuelan equine encephalitis virus infected mouse brain.” Virology Journal 8: 197-197.
38. Shi, Y., E. Mantuano, G. Inoue, W. M. Campana and S. L. Gonias (2009). “Ligand binding to LRP1 transactivates Trk receptors by a Src family kinase-dependent pathway.” Sci Signal 2(68): ra18.
39. Shi, Y., T. Yamauchi, A. Gaultier, S. Takimoto, W. M. Campana and S. L. Gonias (2011). “Regulation of cytokine expression by Schwann cells in response to alpha2-macroglobulin binding to LRP1.” J Neurosci Res 89(4): 544-551.
40. Shiga, Y., A. Shiga, P. Mesci, H. Kwon, C. Brifault, J. H. Kim, J. J. Jeziorski, C. Nasamran, S. Ohtori, A. R. Muotri, S. L. Gonias and W. M. Campana (2019). “Tissue-type plasminogen activator-primed human iPSC-derived neural progenitor cells promote motor recovery after severe spinal cord injury.” Scientific Reports 9(1): 19291.
41. Simon, D., B. Page, M. Vogel, C. Bussmann, C. Blanchard, A. Straumann and H.-U. Simon (2018). “Evidence of an abnormal epithelial barrier in active, untreated and corticosteroid-treated eosinophilic esophagitis.” Allergy 73(1): 239-247.
42. Steele, K. E. and N. A. Twenhafel (2010). “REVIEW PAPER: pathology of animal models of alphavirus encephalitis.” Vet Pathol 47(5): 790-805.
43. Strickland, D. K., S. C. Muratoglu and T. M. Antalis (2011). “Serpin-Enzyme Receptors LDL Receptor-Related Protein 1.” Methods Enzymol 499: 17-31.
44. Subramaniyam, D., P. Glader, K. von Wachenfeldt, J. Burneckiene, T. Stevens and S. Janciauskiene (2006). “C-36 peptide, a degradation product of alpha1-antitrypsin, modulates human monocyte activation through LPS signaling pathways.” Int J Biochem Cell Biol 38(4): 563-575.
45. Taylor, K. G. and S. Paessler (2013). “Pathogenesis of Venezuelan equine encephalitis.” Vet Microbiol 167(1-2): 145-150.
46. Toldo, S., D. Austin, A. G. Mauro, E. Mezzaroma, B. W. Van Tassell, C. Marchetti, S. Carbone, S. Mogelsvang, C. Gelber and A. Abbate (2017). “Low-Density Lipoprotein Receptor-Related Protein-1 Is a Therapeutic Target in Acute Myocardial Infarction.” JACC Basic Transl Sci 2(5): 561-574.
47. Wang, Y., K. Jiang, Q. Zhang, S. Meng and C. Ding (2018). “Autophagy in Negative-Strand RNA Virus Infection.” Frontiers in Microbiology 9(206).
48. Weaver, S. C., C. Ferro, R. Barrera, J. Boshell and J. C. Navarro (2004). “Venezuelan equine encephalitis.” Annu Rev Entomol 49: 141-174.
49. Wujak, L., P. Markart and M. Wygrecka (2016). “The low density lipoprotein receptor-related protein (LRP) 1 and its function in lung diseases.” Histol Histopathol 31(7): 733-745.
50. Wujak, L., J. Schnieder, L. Schaefer and M. Wygrecka (2018). “LRP1: A chameleon receptor of lung inflammation and repair.” Matrix Biology 68-69: 366-381.
51. Yang, L., C.-C. Liu, H. Zheng, T. Kanekiyo, Y. Atagi, L. Jia, D. Wang, A. N'songo, D. Can, H. Xu, X.-F. Chen and G. Bu (2016). “LRP1 modulates the microglial immune response via regulation of JNK and NF-κB signaling pathways.” Journal of Neuroinflammation 13(1): 304.
52. Yang, X., Y. Yu, J. Xu, H. Shu, J. a. Xia, H. Liu, Y. Wu, L. Zhang, Z. Yu, M. Fang, T. Yu, Y. Wang, S. Pan, X. Zou, S. Yuan and Y. Shang (2020). “Clinical course and outcomes of critically ill patients with SARS-CoV-2 pneumonia in Wuhan, China: a single-centered, retrospective, observational study.” The Lancet Respiratory Medicine.
53. Yoon, C., E. A. Van Niekerk, K. Henry, T. Ishikawa, S. Orita, M. H. Tuszynski and W. M. Campana (2013). “Low-density Lipoprotein Receptor-related Protein 1 (LRP1)-dependent Cell Signaling Promotes Axonal Regeneration.” Journal of Biological Chemistry 288(37): 26557-26568.
54. Zlokovic, B. V., R. Deane, A. P. Sagare, R. D. Bell and E. A. Winkler (2010). “Low-density lipoprotein receptor-related protein-1: a serial clearance homeostatic mechanism controlling Alzheimer's amyloid β-peptide elimination from the brain.” Journal of neurochemistry 115(5): 1077-1089.
55. Zurhove, K., C. Nakajima, J. Herz, H. H. Bock and P. May (2008). “Gamma-secretase limits the inflammatory response through the processing of LRP1.” Science signaling 1(47): ra15-ra15.
56. Yamauchi, K., T. Yamauchi, E. Mantuano, K. Murakami, K. Henry, K. Takahashi and W. M. Campana (2013). “Low-density lipoprotein receptor related protein-1 (LRP1)-dependent cell signaling promotes neurotrophic activity in embryonic sensory neurons.” PLoS One 8(9): e75497.
57. Yoon, C., E. A. Van Niekerk, K. Henry, T. Ishikawa, S. Orita, M. H. Tuszynski and W. M. Campana (2013). “Low-density lipoprotein receptor-related protein 1 (LRP1)-dependent cell signaling promotes axonal regeneration.” J Biol Chem 288(37): 26557-26568.
58. Prinz, M., S. Jung and J. Priller (2019). “Microglia Biology: One Century of Evolving Concepts.” Cell 179(2): 292-311.
59. Michell-Robinson, M. A., H. Touil, L. M. Healy, D. R. Owen, B. A. Durafourt, A. Bar-Or, J. P. Antel and C. S. Moore (2015). “Roles of microglia in brain development, tissue maintenance and repair.” Brain 138(Pt 5): 1138-1159.
60. Liu, Y., L. Qin, B. Wilson, X. Wu, L. Qian, A. C. Granholm, F. T. Crews and J. S. Hong (2008). “Endotoxin induces a delayed loss of TH-IR neurons in substantia nigra and motor behavioral deficits.” Neurotoxicology 29(5): 864-870.
61. Cazareth, J., A. Guyon, C. Heurteaux, J. Chabry and A. Petit-Paitel (2014). “Molecular and cellular neuroinflammatory status of mouse brain after systemic lipopolysaccharide challenge: importance of CCR2/CCL2 signaling.” Journal of Neuroinflammation 11(1): 132.
62. Meneses, G., G. Gevorkian, A. Florentino, M. A. Bautista, A. Espinosa, G. Acero, G. Diaz, A. Fleury, I. N. Perez Osorio, A. Del Rey, G. Fragoso, E. Sciutto and H. Besedovsky (2017). “Intranasal delivery of dexamethasone efficiently controls LPS-induced murine neuroinflammation.” Clin Exp Immunol 190(3): 304-314.
63. Zhao, J., W. Bi, S. Xiao, X. Lan, X. Cheng, J. Zhang, D. Lu, W. Wei, Y. Wang, H. Li, Y. Fu and L. Zhu (2019). “Neuroinflammation induced by lipopolysaccharide causes cognitive impairment in mice.” Scientific Reports 9(1): 5790.

1. A SERPIN peptide derivative comprising a core sequence of FVFLM (SEQ ID NO: 1), FVFL[Nle] (SEQ ID NO: 2), PFVFLM (SEQ ID NO: 8), PFVFL[Nle] (SEQ ID NO: 9), and one or more of the following modifications:
(i) a polar head added to the N-terminus of the core sequence, a polar tail added to the C-terminus of the core sequence, or both;
(ii) one or more amino acid residues added to the N-terminus of the core sequence, C-terminus of the core sequence, or both such that the peptide derivative can be cyclized;
(iii) one or more amino acid residues in the core sequence substituted by one or more substitute amino acid residues having less hydrophobicity compared to the one or more amino acid residues in the core sequence that is substituted;
(iv) one or more amino acid residues in the core sequence substituted by one or more substitute amino acid residues having greater hydrophobicity compared to the one or more amino acid residues in the core sequence that is substituted; and
(v) one or more amino acid residues in the core sequence are deleted.

2. The SERPIN peptide derivative of claim 1, wherein the SERPIN peptide derivative is a linear peptide.

3. The SERPIN peptide derivative of claim 1, wherein the SERPIN peptide derivative is a cyclized peptide.

4. The SERPIN peptide derivative of claim 3, wherein the SERPIN peptide derivative is cyclized by forming a disulfide bond between two Cys residues.

5. The SERPIN peptide derivative of claim 3, wherein the SERPIN peptide derivative is cyclized by a linker between two amino acid residues.

6. The SERPIN peptide derivative of claim 1, wherein the polar head or the polar tail comprises two or more charged amino acids such as positively charged amino acids selected from the group consisting of Arg, Lys, and His.

7. The SERPIN peptide derivative of claim 1, wherein the SERPIN peptide derivative has a size of less than 15 amino acid residues.

8. The SERPIN peptide derivative of claim 1, wherein the SERPIN peptide derivative has a size of 7, 8, 9, 10, 11, or 12 amino acid residues.

9. The SERPIN peptide derivative of claim 1, wherein one or more amino acid residues are D-amino acids.

10. A SERPIN peptide derivative comprising the amino acid sequence X1-X2-X3-X4-X5-X6-X7-X8-X9-X10-X11 (SEQ ID NO: 3), wherein:
X1 is a hydrophilic amino acid residue or none;
X2 is a hydrophilic amino acid residue or none;
X3 is a hydrophilic amino acid residue or none;
X4 is a Cys amino acid residue or none;
X5 is a Pro amino acid residue or none;
X6 is a first hydrophobic amino acid residue;
X7 is a short-branched amino acid residue;
X8 is a second hydrophobic amino acid residue;
X9 is a saturated hydrophobic amino acid residue;
X10 is a hydrophilic amino acid of D configuration or Nle; and
X11 is any amino acid residue which allows cyclization of the SERPIN peptide derivative.

11. The SERPIN peptide derivative of claim 10, wherein the first and second hydrophobic amino acid residue are aromatic amino acid residues or Ala or Nal.

12. The SERPIN peptide derivative of claim 10, wherein the short-branched amino acid residue is Val or Thr.

13. The SERPIN peptide derivative of claim 10, wherein the saturated hydrophobic amino acid is Leu.

14. The SERPIN peptide derivative of claim 10, wherein hydrophilic amino acid of D configuration is a D-configured Asp, Glu, Lys, Dap, or Cys residue.

15. The SERPIN peptide derivative of claim 10, wherein
X1 is a basic residue displaying a positive charge or none;
X2 is a basic residue displaying a positive charge or none; and
X3 is a basic residue displaying a positive charge or none.

16. The SERPIN peptide derivative of claim 10, wherein
X1 is a Arg, Lys or His residue or none;
X2 is a Arg, Lys or His residue or none; and
X3 is a Arg, Lys or His residue or none.

17. The SERPIN peptide derivative of claim 10, wherein X6 is Phe or Nal.

18. The SERPIN peptide derivative of claim 10, wherein X8 is Phe or Nal.

19. The SERPIN peptide derivative of claim 10, wherein X6 is Ala, Phe, or Nal, and X4 is Nal.

20. The SERPIN peptide derivative of claim 10, wherein X7 is Asp, Glu, Lys, Dap, or Cys.

21-80. (canceled)


SEQUENCE LISTING (62 sequences)

SEQ ID NO: 1 - protein, 5 aa, synthetic construct
FVFLM

SEQ ID NO: 2 - protein, 5 aa, synthetic construct
  MOD_RES 5: note = Nle
FVFLX

SEQ ID NO: 3 - skipped

SEQ ID NO: 4 - protein, 17 aa, synthetic construct
VKFNKPFVFLMIEQNTK

SEQ ID NO: 5 - protein, 17 aa, synthetic construct
  MOD_RES 11: note = Nle
VKFNKPFVFLXIEQNTK

SEQ ID NO: 6 - protein, 11 aa, synthetic construct
VKFNKPFVFLM

SEQ ID NO: 7 - protein, 4 aa, synthetic construct
FNKP

SEQ ID NO: 8 - protein, 6 aa, synthetic construct
PFVFLM

SEQ ID NO: 9 - protein, 6 aa, synthetic construct
  MOD_RES 6: note = Nle
PFVFLX

SEQ ID NO: 10 - protein, 12 aa, synthetic construct
HHHPFVFLMHHH

SEQ ID NO: 11 - protein, 12 aa, synthetic construct
  MOD_RES 9: note = Nle
HHHPFVFLXHHH

SEQ ID NO: 12 - protein, 12 aa, synthetic construct
  MOD_RES 9: note = Nle
RRRPFVFLXRRR

SEQ ID NO: 13 - protein, 12 aa, synthetic construct
  MOD_RES 9: note = Nle
KKKPFVFLXKKK

SEQ ID NO: 14 - protein, 17 aa, synthetic construct
  MOD_RES 14: note = Nle
EEEVKFNKPFVFLXEEE

SEQ ID NO: 15 - protein, 14 aa, synthetic construct
  MOD_RES 10: note = Nle
RRRCPFVFLXCRRR

SEQ ID NO: 16 - protein, 11 aa, synthetic construct
  MOD_RES 10: note = Nle
RRRCPFVFLXC

SEQ ID NO: 17 - protein, 11 aa, synthetic construct
  MOD_RES 7: note = Nle
CPFVFLXCRRR

SEQ ID NO: 18 - protein, 17 aa, synthetic construct
RRRVKFNKPFVFLMRRR

SEQ ID NO: 19 - protein, 8 aa, synthetic construct
CPFVFLMC

SEQ ID NO: 20 - protein, 8 aa, synthetic construct
  MOD_RES 7: note = Nle
CPFVFLXC

SEQ ID NO: 21 - protein, 11 aa, synthetic construct
RRRCPFVFLMC

SEQ ID NO: 22 - protein, 11 aa, synthetic construct
RRRCPFVFLXC

SEQ ID NO: 23 - protein, 8 aa, synthetic construct
RCFVFLXC

SEQ ID NO: 24 - protein, 9 aa, synthetic construct
  MOD_RES 8: note = Nle
RRCFVFLXC

SEQ ID NO: 25 - protein, 9 aa, synthetic construct
  MOD_RES 8: note = Nle
RRCFVFLXC

SEQ ID NO: 26 - protein, 10 aa, synthetic construct
  MOD_RES 9: note = Nle
RRRCFVFLXC

SEQ ID NO: 27 - protein, 10 aa, synthetic construct
  MOD_RES 9: note = Nle
RRRCFVFTXC

SEQ ID NO: 28 - protein, 10 aa, synthetic construct
  MOD_RES 9: note = Nle
RRRCFTFLXC

SEQ ID NO: 29 - protein, 10 aa, synthetic construct
  MOD_RES 9: note = Nle
RRRCTVFLXC

SEQ ID NO: 30 - protein, 10 aa, synthetic construct
  MOD_RES 9: note = Nle
  VARIANT 7: note = naphylalanine (Nal)
RRRCFVXLXC

SEQ ID NO: 31 - protein, 10 aa, synthetic construct
  VARIANT 7: note = naphylalanine (Nal)
  MOD_RES 9: note = Nle
RRRCXVFLXC

SEQ ID NO: 32 - protein, 10 aa, synthetic construct
  MOD_RES 9: note = Nle
  MOD_RES 10: note = Dpr
RRRDFVFLXX

SEQ ID NO: 33 - protein, 10 aa, synthetic construct
  MOD_RES 10: note = Nle
  MOD_RES 10: note = Dpr
RRRDFVFLXX

SEQ ID NO: 34 - protein, 10 aa, synthetic construct
  MOD_RES 9: note = Nle
  MOD_RES 10: note = Dpr
RRRDFVFLXX

SEQ ID NO: 35 - protein, 10 aa, synthetic construct
  MOD_RES 9: note = Nle
RRREFVFLXK

SEQ ID NO: 36 - protein, 10 aa, synthetic construct
  MOD_RES 9: note = Nle
RRREFVFLXK

SEQ ID NO: 37 - protein, 9 aa, synthetic construct
  MOD_RES 8: note = Nle
RRRFVFLXD

SEQ ID NO: 38 - protein, 10 aa, synthetic construct
  VARIANT 7: note = naphthylalanine (Nal)
  MOD_RES 4: note = Dpr
  MOD_RES 9: note = Nle
RRRXFTXLXD

SEQ ID NO: 39 - protein, 9 aa, synthetic construct
  VARIANT 6: note = naphthylalanine (Nal)
  MOD_RES 8: note = Nle
RRRFTXLXD

SEQ ID NO: 40 - protein, 9 aa, synthetic construct
RRRFVFLAD

SEQ ID NO: 41 - protein, 8 aa, synthetic construct
RRRFVFLD

SEQ ID NO: 42 - protein, 9 aa, synthetic construct
  SITE 8: note = d-serine
RRRFVFLSD

SEQ ID NO: 43 - protein, 9 aa, synthetic construct
  MOD_RES 8: note = Nle
  SITE 7: note = d-serine
RRRFVFSXD

SEQ ID NO: 44 - protein, 9 aa, synthetic construct
  SITE 6: note = d-serine
  MOD_RES 8: note = Nle
RRRFVSLXD

SEQ ID NO: 45 - protein, 9 aa, synthetic construct
  SITE 5: note = d-serine
  MOD_RES 8: note = Nle
RRRFSFLXD

SEQ ID NO: 46 - protein, 9 aa, synthetic construct
  SITE 4: note = d-serine
  MOD_RES 8: note = Nle
RRRSVFLXD

SEQ ID NO: 47 - protein, 8 aa, synthetic construct
  VARIANT 6: note = naphthylalanine (Nal)
RRRFTXLD

SEQ ID NO: 48 - protein, 8 aa, synthetic construct
  SITE 1: note = reverse lysine
  VARIANT 6: note = naphthylalanine (Nal)
KRRFTXLD

SEQ ID NO: 49 - protein, 8 aa, synthetic construct
  SITE 1: note = reverse arginine
  VARIANT 6: note = naphthylalanine (Nal)
RRRFTXLD

SEQ ID NO: 50 - protein, 7 aa, synthetic construct
  SITE 1: note = reverse arginine
  SITE 2: note = reverse arginine
  VARIANT 5: note = naphthylalanine (Nal)
RRFTXLD

SEQ ID NO: 51 - protein, 9 aa, synthetic construct
  MOD_RES 4: note = Dpr
  VARIANT 7: note = naphthylalanine (Nal)
RRRXFTXLD

SEQ ID NO: 52 - protein, 8 aa, synthetic construct
  MOD_RES 3: note = Dpr
  VARIANT 6: note = naphthylalanine (Nal)
RRXFTXLD

SEQ ID NO: 53 - protein, 9 aa, synthetic construct
  VARIANT 7: note = naphthylalanine (Nal)
RRRKFTXLD

SEQ ID NO: 54 - skipped

SEQ ID NO: 55 - protein, 9 aa, synthetic construct
  MOD_RES 3: note = Dpr
  VARIANT 6: note = naphthylalanine (Nal)
  SITE 8: note = d-serine
RRXFTXLSD

SEQ ID NO: 56 - protein, 9 aa, synthetic construct
  SITE 8: note = d-Dpr
RRRFVFLXD

SEQ ID NO: 57 - protein, 9 aa, synthetic construct
  SITE 8: note = d-lysine
RRRFVFLKD

SEQ ID NO: 58 - protein, 9 aa, synthetic construct
  SITE 8: note = d-aspartic acid
RRRFVFLDD

SEQ ID NO: 59 - protein, 10 aa, synthetic construct
  VARIANT 7: note = naphthylalanine (Nal)
  MOD_RES 9: note = Nle
RRRFTFXAXD

SEQ ID NO: 60 - protein, 9 aa, synthetic construct
  MOD_RES 8: note = Nle
  VARIANT 6: note = naphthylalanine (Nal)
RRRATXLXD

SEQ ID NO: 61 - protein, 8 aa, synthetic construct
  VARIANT 6: note = naphthylalanine (Nal)
  SITE 7: note = d-serine
RRRFTLSD

SEQ ID NO: 62 - protein, 9 aa, synthetic construct
  SITE 8: note = d-serine
RRRFTWLSD